(19) United States
(12) Patent Application Publication
(10) Pub. No.: US 2026/0096784 A1
(43) Pub. Date: Apr. 9, 2026

(54) ANTICIPATING AND INTERVENING IN NOCTURNAL HYPOGLYCEMIC EPISODES

(71) Applicant: Oregon Health & Science University, Portland, OR (US)

(72) Inventors: Clara M. MOSQUERA-LOPEZ, Portland, OR (US); Peter G. JACOBS, Portland, OR (US); Leah WILSON, Portland, OR (US)

(21) Appl. No.: 19/354,725

(22) Filed: Oct. 9, 2025

Related U.S. Application Data
(60) Provisional application No. 63/705,499, filed on Oct. 9, 2024.

(51) Int. Cl. A61B 5/00; A61B 5/145

(52) U.S. Cl. CPC A61B 5/7275; A61B 5/14532; A61B 5/742; A61B 5/7475

(57) ABSTRACT

A computer-implemented method and system reduce the likelihood of nocturnal hypoglycemia by predicting overnight glucose level and providing personalized recommendations. Glucose-derived features from continuous glucose monitor (CGM) data, physical-activity features, and demographic features are extracted and input into a neural network configured to output parameters of a probability distribution representing a predicted minimum overnight glucose value and an associated predictive uncertainty. Based on the network output, a probability that the glucose level will fall below a specified hypoglycemic level within an upcoming sleep period is computed and compared to a risk threshold. When the probability meets or exceeds the risk threshold, a mobile computing device displays a personalized bedtime snack recommendation selected to mitigate the predicted hypoglycemia event. The approach integrates physiological data, individualized modeling, and decision support to provide actionable guidance for avoiding nocturnal hypoglycemia.

### RELATED APPLICATION

This application claims priority benefit of U.S. Provisional Patent Application No. 63/705,499 filed Oct. 9, 2024, which is hereby incorporated by reference.

### ACKNOWLEDGEMENT OF GOVERNMENT SUPPORT

This invention was made with government support under R21 DK128582 awarded by the National Institutes of Health. The government has certain rights in the invention.

### TECHNICAL FIELD

This disclosure relates to systems and methods for predicting and mitigating possible nocturnal hypoglycemia events in persons having type 1 diabetes (T1D) who are using multiple daily injection (MDI) for glucose control and who are equipped with continuous glucose monitor (CGM) systems.

### BACKGROUND INFORMATION

Nocturnal hypoglycemia accounts for more than 50% of level-two hypoglycemia events (glucose <54 mg/dL) in persons with T1D.^{1,2} Increased levels of late day physical activity (PA) dramatically increase risk for nocturnal hypoglycemia.³ However, this risk can be difficult to predict because of individual variation in whole body insulin sensitivity post-exercise⁴ and the variability in the blunting of the counterregulatory responses to ensuing hypoglycemia after exercise, which may be impacted by several variables such as recent hypoglycemia events, sex, and the intensity of exercise.⁵⁻⁸ Even with the use of intermittently scanned or real-time CGM, nocturnal hypoglycemia can be difficult to manage clinically as individuals are unlikely to recognize symptoms while sleeping and may not awaken to hypoglycemia alarms from CGM systems.^{9,10}

Although advanced insulin therapies are now available, most people with T1D continue to use MDI, with or without CGM.^{11,12} A recent study showed that MDI users using CGM spent more time with glucose <54 mg/dL and experienced more nocturnal hypoglycemia than standard pump or hybrid closed-loop (HCL) users.¹²

Parallel approaches have emerged to separately prevent or predict nocturnal hypoglycemia. Guidelines recommend modifications to insulin doses and carbohydrate consumption without bolus insulin administration following exercise to help lower the risk of post-exercise nocturnal hypoglycemia,¹³⁻¹⁵ but these guidelines rely heavily on user experiences, their perceived risk for nocturnal hypoglycemia, and actions to make adjustments within the appropriate time period. While there have been several publications demonstrating methods for predicting nocturnal hypoglycemia,¹⁶⁻²³ these algorithms do not estimate the risk and timing of nocturnal hypoglycemia relative to exercise based on known risk factors (e.g., antecedent exercise intensity, duration, and recent glucose trends) nor do they provide predictive uncertainty. Thus, there remains a need for a system that lowers the patient burden for glycemic self-management by both predicting nocturnal hypoglycemia and suggesting prophylactic carbohydrate consumption at bedtime after a predominantly sedentary day or an active day.

### SUMMARY OF THE DISCLOSURE

In one aspect, a computer-implemented method for reducing likelihood of a person experiencing nocturnal hypoglycemia comprises: extracting a plurality of features including glucose-derived features from CGM data, activity-derived features from physical-activity data, and demographic-derived features associated with the person; inputting the plurality of features into a neural network configured to output parameters of a probability distribution representing both a predicted minimum overnight glucose value and an associated predictive uncertainty; determining, based on output of the neural network, whether a probability that the person's glucose level will fall below a specified hypoglycemic level within a specified time period during an upcoming sleep session meets or exceeds a predetermined risk threshold; and in response to determining that the probability meets or exceeds the predetermined risk threshold indicative of a predicted hypoglycemia event, causing a display, on a user interface of a mobile computing device, of a personalized bedtime snack recommendation configured to allow the person to avoid the predicted hypoglycemia event. The method further comprises receiving, via the user interface, an input indicating whether the person consumed a recommended snack, and logging the input in a user profile stored in memory of the mobile computing device or of a remote server. In the method, the neural network includes an input layer configured to receive the plurality of features, one or more hidden layers, and an output layer configured to generate parameters of a normal inverse-gamma distribution. In the method, the neural network is trained to output parameters (γ, ν, α, β) of the normal inverse-gamma distribution for characterizing both the predicted minimum overnight glucose value and the associated predictive uncertainty. In the method, the plurality of features includes at least one of: a glucose-trend feature calculated over the hour preceding bedtime; a rate-of-change feature; or an activity-intensity feature derived from accelerometer data. In the method, the specified time period comprises a temporal window corresponding to an early-night window of approximately 0 to 4 hours after bedtime or a late-night window of approximately 4 to 8 hours after bedtime. In the method, the personalized bedtime snack recommendation is for a fast-absorbing snack, comprising simple carbohydrates, selected for the early-night window. In the method, the personalized bedtime snack recommendation is for a slow-absorbing snack comprising complex carbohydrates, protein, and fat selected for the late-night window. In the method, the slow-absorbing snack has an approximate 4:2:1 ratio of complex carbohydrates, protein, and fat, with 1-2 grams of dietary fiber. The method further comprises identifying the personalized bedtime snack recommendation having a carbohydrate content between approximately 15 grams and 30 grams based on the predicted minimum overnight glucose value. In the method, the personalized bedtime snack recommendation further specifies a portion size selected based on the predicted minimum glucose value. The method further comprises receiving, by the mobile computing device, the CGM data acquired from a glucose sensor worn by the person with diabetes. The method further comprises receiving, by the mobile computing device, self-reported physical-activity data entered by the person through the user interface of a smartphone application. The method further comprises receiving, by the mobile computing device, monitored physical-activity data acquired automatically by a wearable fitness tracker worn by the person and communicatively coupled with the mobile computing device. The method further comprises computing a early-night probability that the person's glucose level will fall below the specified hypoglycemic level during an early-night window corresponding to approximately 0 to 4 hours after bedtime and a late-night probability that the person's glucose level will fall below the specified hypoglycemic level during a late-night window corresponding to approximately 4 to 8 hours after bedtime. The method further comprises comparing each of the early-night probability and the late-night probability to corresponding predetermined risk thresholds, and determining that a predicted hypoglycemia event will occur when either of the probabilities meets or exceeds its corresponding predetermined risk threshold. The method further comprises identifying a corresponding bedtime snack recommendation based on which of the probabilities meets or exceeds its corresponding predetermined risk threshold. In the method, a fast-absorbing snack comprising simple carbohydrates is selected when the early-night probability meets or exceeds its threshold, and a slow-absorbing snack comprising complex carbohydrates, protein, and fat is selected when the late-night probability meets or exceeds its threshold. In the method, the predetermined risk threshold for the early-night window is greater than the predetermined risk threshold for the late-night window to account for a higher physiological sensitivity to early-night glucose decline. In the method, the predetermined risk thresholds for the early-night and late-night windows are independently defined based on physical-activity level and timeframe. In the method, the neural-network output includes different probability distribution parameter values for each window, and the early-night and late-night probabilities are computed from corresponding parameter values.

In another aspect, a non-transitory computer-readable storage medium storing instructions that, when executed by one or more processors of a computing device, configure the computing device to: extract a plurality of features including glucose-derived features from CGM data, activity-derived features from physical-activity data, and demographic-derived features associated with a person; input the plurality of features into a neural network configured to output parameters of a probability distribution representing both a predicted minimum overnight glucose value and an associated predictive uncertainty; determine, based on output of the neural network, whether a probability that the person's glucose level will fall below a specified hypoglycemic level within a specified time period during an upcoming sleep session meets or exceeds a predetermined risk threshold; and in response to determining that the probability meets or exceeds the predetermined risk threshold indicative of a predicted hypoglycemia event, cause a display, on a user interface of a mobile computing device, of a personalized bedtime snack recommendation configured to allow the person to avoid the predicted hypoglycemia event. The non-transitory computer-readable storage medium stores instructions that further configure the computing device to receive, via the user interface, an input indicating whether the person consumed a recommended snack, and to log the input in a user profile stored in a memory of the mobile computing device or of a remote server. The non-transitory computer-readable storage medium stores instructions in which the neural network comprises an input layer configured to receive the plurality of features, one or more hidden layers, and an output layer configured to generate parameters of a normal inverse-gamma distribution. The non-transitory computer-readable storage medium stores instructions in which the neural network is trained to output parameters (γ, ν, α, β) of the normal inverse-gamma distribution characterizing both the predicted minimum overnight glucose value and the associated predictive uncertainty. The non-transitory computer-readable storage medium stores instructions in which the plurality of features includes at least one of: (a) a glucose-trend feature calculated over the hour preceding bedtime; (b) a rate-of-change feature; or (c) an activity-intensity feature derived from manually entered or monitored activity data. The non-transitory computer-readable storage medium stores instructions in which the specified time period comprises a temporal window corresponding to an early-night window of approximately 0 to 4 hours after bedtime or a late-night window of approximately 4 to 8 hours after bedtime. The non-transitory computer-readable storage medium stores instructions that further configure the computing device to compute an early-night probability that the person's glucose level will fall below the specified hypoglycemic level during the early-night window and a late-night probability that the person's glucose level will fall below the specified hypoglycemic level during the late-night window. The non-transitory computer-readable storage medium stores instructions that further configure the computing device to compare each of the early-night probability and the late-night probability to corresponding predetermined risk thresholds, and to determine that a predicted hypoglycemia event will occur when either of the probabilities meets or exceeds its corresponding predetermined risk threshold. The non-transitory computer-readable storage medium stores instructions that further configure the computing device to identify a corresponding bedtime snack recommendation based on which of the probabilities meets or exceeds its corresponding predetermined risk threshold, in which (a) a fast-absorbing snack comprising simple carbohydrates is selected when the early-night probability meets or exceeds its threshold, and (b) a slow-absorbing snack comprising complex carbohydrates, protein, and fat is selected when the late-night probability meets or exceeds its threshold. The non-transitory computer-readable storage medium stores instructions in which the predetermined risk thresholds for the early-night and late-night windows are independently defined based on physical-activity level and timeframe. The non-transitory computer-readable storage medium stores instructions that further configure the computing device to identify the personalized bedtime snack recommendation having a carbohydrate content between approximately 15 grams and 30 grams based on the predicted minimum overnight glucose value. The non-transitory computer-readable storage medium stores instructions that further configure the computing device to specify a portion size of the personalized bedtime snack recommendation based on the predicted minimum overnight glucose value. The non-transitory computer-readable storage medium stores instructions that further configure the computing device to receive CGM data acquired from a glucose sensor worn by the person with diabetes. The non-transitory computer-readable storage medium stores instructions that further configure the computing device to receive self-reported physical-activity data entered by the person through the user interface of a smartphone application. The non-transitory computer-readable storage medium stores instructions that further configure the computing device to receive monitored physical-activity data acquired automatically by a wearable fitness tracker worn by the person and communicatively coupled with the computing device. The non-transitory computer-readable storage medium stores instructions that further configure the computing device to determine that a predicted hypoglycemia event will occur when either the early-night probability or the late-night probability meets or exceeds its corresponding predetermined risk threshold. The non-transitory computer-readable storage medium stores instructions that further configure the computing device to generate an alert when either probability meets or exceeds its corresponding predetermined risk threshold and to present the alert together with the bedtime snack recommendation on the user interface. The non-transitory computer-readable storage medium stores instructions in which the slow-absorbing snack has an approximate 4:2:1 ratio of complex carbohydrates, protein, and fat, with 1-2 grams of dietary fiber. The non-transitory computer-readable storage medium stores instructions that further configure the computing device to compute the early-night and late-night probabilities using different parameter values of the probability distribution output by the neural network. The non-transitory computer-readable storage medium stores instructions that further configure the computing device to adapt the predetermined risk thresholds for the early-night and late-night windows according to at least one of the person's physical-activity level or the timeframe. The non-transitory computer-readable storage medium stores instructions in which execution of the instructions by the computing device further causes extraction of glucose-trend, rate-of-change, and activity-intensity features and computation of corresponding probabilities for early-night and late-night windows to provide proactive recommendations for reducing nocturnal hypoglycemia.

In another aspect, a system comprises a processor and a memory storing instructions that, when executed by the processor, cause the system to: extract a plurality of features including glucose-derived features from continuous glucose monitor (CGM) data, activity-derived features from physical-activity data, and demographic-derived features associated with a person; input the plurality of features into a neural network configured to output parameters of a probability distribution representing both a predicted minimum overnight glucose value and an associated predictive uncertainty; determine, based on output of the neural network, whether a probability that the person's glucose level will fall below a specified hypoglycemic level within a specified time period during an upcoming sleep session meets or exceeds a predetermined risk threshold; and in response to determining that the probability meets or exceeds the predetermined risk threshold indicative of a predicted hypoglycemia event, cause a display, on a user interface of a mobile computing device, of a personalized bedtime snack recommendation configured to allow the person to avoid the predicted hypoglycemia event. The system is such that the instructions further cause the processor to receive, via the user interface, an input indicating whether the person consumed a recommended snack, and to log the input in a user profile stored in a memory of the mobile computing device or of a remote server. In the system, the neural network comprises an input layer configured to receive the plurality of features, one or more hidden layers, and an output layer configured to generate parameters of a normal inverse-gamma distribution. In the system, the neural network is trained to output parameters (γ, ν, α, β) of the normal inverse-gamma distribution characterizing both the predicted minimum overnight glucose value and the associated predictive uncertainty. In the system, the plurality of features includes at least one of: (a) a glucose-trend feature calculated over the hour preceding bedtime; (b) a rate-of-change feature; or (c) an activity-intensity feature derived from manually entered or monitored activity data. In the system, the specified time period comprises a temporal window corresponding to an early-night window of approximately 0 to 4 hours after bedtime or a late-night window of approximately 4 to 8 hours after bedtime. In the system, the processor is further configured to compute an early-night probability that the person's glucose level will fall below the specified hypoglycemic level during the early-night window and a late-night probability that the person's glucose level will fall below the specified hypoglycemic level during the late-night window. In the system, the processor is further configured to compare each of the early-night probability and the late-night probability to corresponding predetermined risk thresholds, and to determine that a predicted hypoglycemia event will occur when either of the probabilities meets or exceeds its corresponding predetermined risk threshold. In the system, the processor is further configured to identify a corresponding bedtime snack recommendation based on which of the probabilities meets or exceeds its corresponding predetermined risk threshold, in which (a) a fast-absorbing snack comprising simple carbohydrates is selected when the early-night probability meets or exceeds its threshold, and (b) a slow-absorbing snack comprising complex carbohydrates, protein, and fat is selected when the late-night probability meets or exceeds its threshold. In the system, the predetermined risk thresholds for the early-night and late-night windows are independently defined based on physical-activity level and timeframe. In the system, the processor is further configured to identify the personalized bedtime snack recommendation having a carbohydrate content between approximately 15 grams and 30 grams based on the predicted minimum overnight glucose value. In the system, the processor is further configured to specify a portion size of the personalized bedtime snack recommendation based on the predicted minimum overnight glucose value. In the system, the processor is further configured to receive CGM data acquired from a glucose sensor worn by the person with diabetes. In the system, the processor is further configured to receive self-reported physical-activity data entered by the person through the user interface of a smartphone application. In the system, the processor is further configured to receive monitored physical-activity data acquired automatically by a wearable fitness tracker worn by the person and communicatively coupled with the mobile computing device. In the system, the processor is further configured to determine that a predicted hypoglycemia event will occur when either the early-night probability or the late-night probability meets or exceeds its corresponding predetermined risk threshold. In the system, the processor is further configured to generate an alert when either probability meets or exceeds its corresponding predetermined risk threshold and to present the alert together with the bedtime snack recommendation on the user interface. In the system, the slow-absorbing snack has an approximate 4:2:1 ratio of complex carbohydrates, protein, and fat, with 1-2 grams of dietary fiber. In the system, the processor is further configured to compute the early-night and late-night probabilities using different parameter values of the probability distribution output by the neural network. In the system, the processor is further configured to adapt the predetermined risk thresholds for the early-night and late-night windows according to at least one of the person's physical-activity level or the timeframe. In the system, execution of the instructions by the processor further causes extraction of glucose-trend, rate-of-change, and activity-intensity features and computation of corresponding probabilities for early-night and late-night windows to provide proactive recommendations for reducing nocturnal hypoglycemia.

In one aspect, a system comprises a CGM sensor configured to be worn by a person and to generate CGM data, the CGM sensor including a wireless transmitter; a mobile computing device comprising a processor, a memory, a wireless communication interface, and a user-interface display; and at least one of (i) the mobile computing device executing program instructions or (ii) a remote prediction server communicatively coupled to the mobile computing device and executing program instructions. The program instructions are configured to cause (1) the mobile computing device to receive the CGM data from the CGM sensor; (2) the mobile computing device to acquire physical-activity data comprising at least one of self-reported physical-activity data entered via the user interface and monitored physical-activity data obtained from a wearable fitness tracker communicatively coupled with the mobile computing device, and to store demographic data associated with the person; (3) the processor of the mobile computing device or the remote prediction server to extract a plurality of features including glucose-derived features from the CGM data, activity-derived features from the physical-activity data, and demographic-derived features from the demographic data; (4) the processor of the mobile computing device or the remote prediction server to input the plurality of features into a neural network configured to output parameters of a probability distribution representing both a predicted minimum overnight glucose value and an associated predictive uncertainty; (5) the processor of the mobile computing device or the remote prediction server to determine whether a probability that the person's glucose level will fall below a specified hypoglycemic level within a specified time period during an upcoming sleep session meets or exceeds a predetermined risk threshold; and (6) the mobile computing device to cause a display of a personalized bedtime snack recommendation on the user-interface display in response to determining that the probability meets or exceeds the predetermined risk threshold, the recommendation being configured to allow the person to avoid a predicted hypoglycemia event.

The system further comprises a CGM receiver distinct from the mobile computing device and configured to receive the CGM data from the CGM sensor, wherein the mobile computing device is configured to receive the CGM data from the CGM receiver. In some embodiments, the remote prediction server executes the neural network and transmits a prediction result to the mobile computing device, and the mobile computing device generates the personalized bedtime snack recommendation based on the prediction result. In some embodiments, the mobile computing device executes the neural network locally and generates the prediction result without transmitting the plurality of features to a remote server.

The specified time period comprises a temporal window corresponding to an early-night window of approximately 0 to 4 hours after bedtime or a late-night window of approximately 4 to 8 hours after bedtime. The processor of the mobile computing device or the remote prediction server is further configured to compute an early-night probability that the person's glucose level will fall below the specified hypoglycemic level during the early-night window and a late-night probability that the person's glucose level will fall below the specified hypoglycemic level during the late-night window. The processor is further configured to compare each of the early-night probability and the late-night probability to corresponding predetermined risk thresholds, and to determine that a predicted hypoglycemia event will occur when either of the probabilities meets or exceeds its corresponding predetermined risk threshold. The mobile computing device is configured to identify a corresponding bedtime snack recommendation based on which of the probabilities meets or exceeds its corresponding predetermined risk threshold, wherein (a) a fast-absorbing snack comprising simple carbohydrates is selected when the early-night probability meets or exceeds its threshold, and (b) a slow-absorbing snack comprising complex carbohydrates, protein, and fat is selected when the late-night probability meets or exceeds its threshold. The slow-absorbing snack has an approximate 4:2:1 ratio of complex carbohydrates, protein, and fat, with 1-2 grams of dietary fiber. The mobile computing device is configured to identify the personalized bedtime snack recommendation having a carbohydrate content between approximately 15 grams and 30 grams based on the predicted minimum overnight glucose value, and to specify a portion size of the personalized bedtime snack recommendation based on the predicted minimum overnight glucose value.

The mobile computing device is configured to receive self-reported physical-activity data entered by the person through the user interface of a smartphone application, and the system further comprises the wearable fitness tracker configured to provide monitored physical-activity data to the mobile computing device via a wireless link, the monitored physical-activity data being included in the activity-derived features. The mobile computing device is configured to receive the CGM data wirelessly from the CGM sensor via the wireless communication interface and to store the CGM data in the memory. The mobile computing device is further configured to generate an alert when either probability meets or exceeds its corresponding predetermined risk threshold and to present the alert together with the bedtime snack recommendation on the user-interface display. The mobile computing device is configured to receive, via the user interface, an input indicating whether the person consumed a recommended snack, and to log the input in a user profile stored in a memory of the mobile computing device or of a remote server. The processor is further configured to compute the early-night and late-night probabilities using different parameter values of the probability distribution output by the neural network for respective temporal windows. The predetermined risk thresholds for the early-night and late-night windows are independently defined based on physical-activity level and timeframe. Execution of the program instructions causes extraction of glucose-trend, rate-of-change, and activity-intensity features and computation of corresponding probabilities for early-night and late-night windows to provide proactive recommendations for reducing nocturnal hypoglycemia.

Additional aspects and advantages will be apparent from the following detailed description of embodiments, which proceeds with reference to the accompanying drawings.

### BRIEF DESCRIPTION OF THE DRAWINGS

To easily identify the discussion of any particular element or act, the most significant digit or digits in a reference number refer to the figure number in which that element is first introduced.

FIG. 1 is an annotated block diagram showing an example Nocturnal Hypoglycemia Alert and Intervention System (NHAIS) configured to predict probability of nocturnal hypoglycemia and prescribe bedtime snacks to help reduce exposure to overnight low glucose, in accordance with one embodiment.

FIG. 2 is an annotated block diagram overview of recommender system with a prediction algorithm.

FIG. 3 is an annotated block diagram showing an example of a fully connected evidential neural network (ENN) for the prediction algorithm in FIG. 1.

FIG. 4 is a flow diagram showing an example procedure for training an ENN model.

FIG. 5 is a flow diagram showing an example procedure for testing and making predictions using the trained ENN model.

FIG. 6 is a bar chart showing the features used to train the disclosed machine-learning algorithm, ranked by a mutual information criterion.

FIG. 7 is a graph showing the association between prediction root-mean-square error (RMSE) and evidential uncertainty for a training and a testing dataset.

FIG. 8 is a box plot showing predicted probability of nocturnal hypoglycemia stratified by portion of the night.

FIG. 9 is a box plot showing predicted probability of nocturnal hypoglycemia stratified by active versus sedentary days.

FIG. 10A and FIG. 10B are, respectively, left- and right-sides of a flow diagram of a process for predicting, for a person using a NHAIS (FIG. 1), the likelihood of experiencing a nocturnal hypoglycemic event and whether an alert and snack recommendation should be presented.

FIG. 11A, FIG. 11B, FIG. 11C, and FIG. 11D (collectively, FIG. 11) are a series of user interface displays presented on a smart device, showing operation of a software application providing a snack recommendation.

FIG. 12 is an annotated timing diagram of a clinical application of an evidential regression-based decision support tool integrated into a mobile health application, in accordance with one embodiment.

FIG. 13 is a block diagram of a cloud-connected diabetes management system in accordance with one embodiment.

FIG. 14 is a block diagram of example computing components for the NHAIS (FIG. 1), the recommender system (FIG. 2), the cloud-connected diabetes management system (FIG. 13), or any associated devices in accordance with some embodiments.

### DETAILED DESCRIPTION OF EMBODIMENTS

Disclosed herein are systems and methods for prediction and prevention of nocturnal hypoglycemia. Various embodiments employ an algorithm that predicts, at bedtime, the probability of a future hypoglycemic event and provides an estimated time frame for its occurrence and a measure of uncertainty. This information may be further passed to a Smart Snack algorithm to recommend carbohydrate intake at bedtime. This system aims to help people with T1D on MDI to avoid nocturnal hypoglycemia by reducing both the frequency and duration of adverse hypoglycemic events.

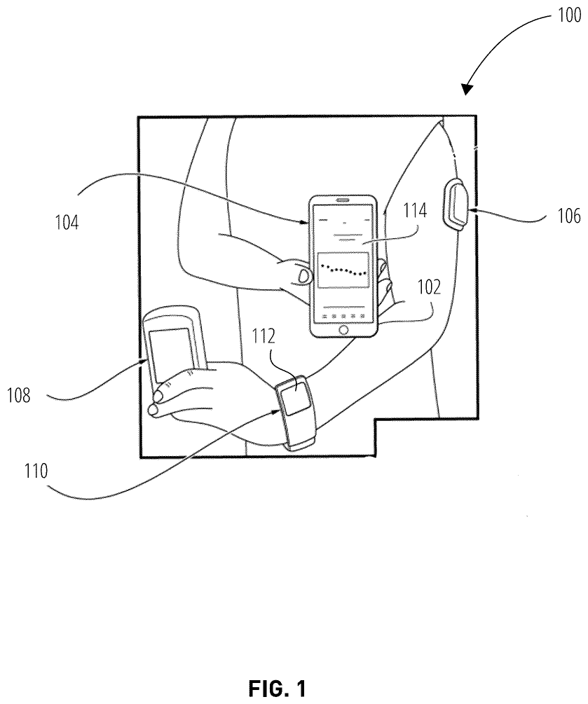

FIG. 1 shows a Nocturnal Hypoglycemia Alert and Intervention System (NHAIS) 100. In this example, NHAIS 100 includes a personalized computing device 102 configured (e.g., using a smartphone app 104) to predict the probability of a hypoglycemic episode in a specified time period during an upcoming sleep session, and, if necessary, provide an alert to the user and prescribe a proactive carbohydrate intervention (i.e., a snack) to obviate the predicted hypoglycemic episode.

Computing device 102 may include a mobile phone, tablet device, or server. In the example of FIG. 1, computing device 102 is depicted as a smartphone configured to communicate with a wearable glucose sensing medical device 106 such as a disposable CGM sensor (which may also function as a transmitter) and, optionally, an associated CGM receiver 108 (or similar device configured to implement features for glucose monitoring).

Also shown in FIG. 1 is a physical activity sensor 110 in the form of a smart watch 112 for quantifying physical activity and communicating that activity to computing device 102. In alternate embodiments, physical activity may be measured using a body-attached fitness tracker, a heart rate monitoring device, an exercise machine such as a treadmill or stationary bicycle, or self-reported by user entry. Alerts and prescribed carbohydrate interventions can be presented to the user visually on a display screen 114 of computing device 102 (e.g., displayed by running smartphone app 104 or provided as a push notification). Alerts may also be communicated by auditory output or tactile stimulation from a suitably configured physical activity sensor 110, computing device 102, or other hardware in communication with NHAIS 100.

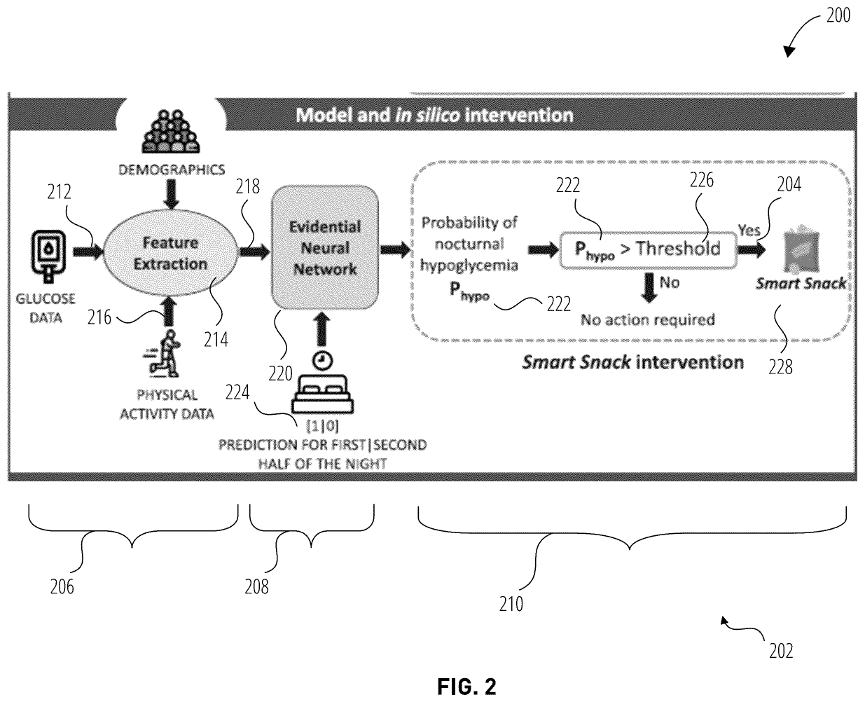

FIG. 2 shows a schematic overview of a carbohydrate snack recommender system 200 (also referred to as a “Smart Snack system”) employs a prediction algorithm 202 to generate an alert 204, e.g., in the form of a recommended Smart Snack (or simply “Smart Snack”) before bedtime to reduce risk of a nocturnal hypoglycemia event. In this example, prediction algorithm 202 includes dataset feature extraction and selection 206, a trained evidential neural network (ENN) model 208, and a so-called Smart Snack intervention recommendation algorithm 210. Each of these aspects is described in detail below.

As shown, dataset feature extraction and selection 206 is configured to receive as input three forms of data: (1) glucose data 212 (provided, for example, by a CGM system coupled to a user), (2) demographic data 214 (for example, age and sex of the user), and (3) physical activity data 216 (provided, for example, by a fitness tracker, pedometer, heart rate monitor, or the like, to allow recommender system 200 to determine whether the user has been predominantly sedentary or active over the course of a day).

Datasets: Free-living data from 366 individuals from the T1DEXI Study and Glooko Inc. (Mountain View, CA, USA) (refer to Table 1 for details) were used. The T1DEXI Study is a large real-world observational study conducted at-home, involving the collection of glucose management data from 497 people with T1D to study the effects of different types of exercise (i.e., cardio, interval, and strength) on glycemic control. Participants from the T1DEXI Study were recruited from around the United States. An Institutional Review Board approved the T1DEXI Study and electronic informed consent was obtained from each participant. The study had two phases: the initial pilot data collection²⁴ followed by the subsequent main data collection.²⁵ The T1DEXI dataset included data from physically active adults (mean age 37±14 years; HbA1c 6.6±0.8% [49±8.7 mmol/mol]) on MDI, standard insulin pump, or HCL therapies, who wore an unblinded CGM and a fitness tracker during 4 weeks. In brief, participants performed randomly assigned structured exercise sessions and reported PA, food intake, and insulin dosages (in the case of MDI users). The outcomes of the study included the change in glucose during exercise and differences in time in rage 70-180 mg/dL between physically active versus sedentary days.²⁵

TABLE 1
Description of datasets used for model training and testing

| | Dataset | | |
|---|---|---|---|
| | T1DEXI STUDY | | |
| CHARACTERISTIC | PILOT STUDY | MAIN STUDY | GLOOKO |
| PARTICIPANTS, N | 7 | 88 | 271 |
| NIGHTS WITH DATA, N | 210 | 2278 | 41 666 |
| PHYSICAL ACTIVITY DATA | YES | YES | NO |
| DEMOGRAPHICS | | | |
| BIOLOGICAL SEX | 2/5/0 | 55/33/0 | 79/66/126 |
| (FEMALE/MALE/UNKNOWN), N | | | |
| AGE (MEAN ± SD) AT BASELINE, YEARS | 28 ± 11 | 38 ± 14 | 26 ± 18 |

OVERALL GLUCOSE CONTROL, AVERAGE
[RANGE] AT THE PARTICIPANT LEVEL

| | | | |
|---|---|---|---|
| TIME IN TARGET RANGE 70-180 MG/DL, % | 52.7 | 72.3 | 59.2 |
| | [38.1, 66.4] | [26.5, 98.7] | [2.6, 100.0] |
| TIME ABOVE RANGE >180 MG/DL, % | 37.3 | 24.0 | 39.1 |
| | [21.3, 55.1] | [0.0, 73.5] | [0.0, 97.4] |
| TIME BELOW RANGE <70 MG/DL, % | 10.0 | 3.7 | 1.7 |
| | [1.3, 15.7] | [0.0, 15.8] | [0.0, 15.4] |

OVERNIGHT HYPOGLYCEMIA (11:00 pm-7:00 am),
AVERAGE [RANGE] AT THE PARTICIPANT LEVEL

| | | | |
|---|---|---|---|
| PROBABILITY OF NOCTURNAL | 44.8 | 24.3 | 16.2 |
| HYPOGLYCEMIA, % | | | |
| TIME IN OVERNIGHT GLUCOSE <70 | 15.1 | 3.9 | 2.1 |
| MG/DL, % | [2.1, 33.3] | [0.0, 18.5] | [0.0, 53.8] |
| TIME IN OVERNIGHT GLUCOSE <70 | 73 | 18 | 9 |
| MG/DL, MIN | [10, 160] | [0, 80] | [0, 94] |
| TIME IN OVERNIGHT GLUCOSE <54 | 7.2 | 1.0 | 0.4 |
| MG/DL, % | [0.9, 18.2] | [0.0, 10.0] | [0.0, 8.1] |
| TIME IN OVERNIGHT GLUCOSE <54 | 34 | 5 | 2 |
| MG/DL, MIN | [5, 88] | [0, 44] | [0, 39] |

Although the T1DEXI dataset contains data from participants on various insulin treatment modalities; in the example presented here, data from participants on MDI therapy (N=7 from the pilot phase and N=88 from the main phase) was exclusively employed as this group represents the target population for the prediction-based intervention described herein.

The Glooko dataset comprises glucose and insulin data from MDI users (N=271) who lived in the United States. Demographic data in the Glooko dataset were limited to age and biological sex. Glooko provided the de-identified dataset for the study with required consent from patients and healthcare providers.

For algorithm development and testing, the participants from the T1DEXI and Glooko datasets were split in an 80:20 ratio such that 80% of the data was used only for model development while the remaining 20% was used for testing. The T1DEXI dataset included labeled PA and sleep data. Distribution of bedtimes from the T1DEXI Study was used to estimate bedtime in the Glooko dataset. There were no insulin boluses reported in the eight hours after the estimated bedtimes.

These glucose data 212, demographic data 214, and physical activity data 216 are passed to a feature extractor 218 process stored and executed on a computing device, such that the data are parsed into a set of features 220.

Feature Extraction and Selection: Glucose features were derived from CGM data (e.g., glucose statistics, low/high blood glucose index, continuous overlapping net glycemic action, 26 percentage time in clinically relevant glucose ranges,²⁷ and long continuous glucose rises or drops) and calculated across different time frames prior to bedtime (i.e., previous seven nights, daytime [7:00 am to bedtime], 1-24 h, and 30 min) and at bedtime. A maximum allowable threshold of 30% missing CGM data was employed during feature calculation to ensure that each participant had sufficient data represented in the dataset. Data in the training and test sets were well balanced and had equivalent distributions to mitigate the influence of potential outliers.

Agglomerative clustering²⁸ was used for feature selection. Glucose features were clustered based on the features' pairwise mutual information (MI),¹² such that correlated features were grouped together. The optimal number of clusters was determined to be 69 by maximizing the Silhouette score.²⁹ From each cluster, the feature with maximum MI with nocturnal hypoglycemia was included in the model. Other widely used approaches such as Lasso³⁰ could have been used for feature selection. However, Lasso can perform erratically when features are correlated.³¹

Missing values of glucose features and the duration of PA were imputed with the median value of the features in the training dataset and standardization was used for feature scaling. Scaling of continuous features was done by subtracting the feature mean from the unscaled feature value and then dividing by the feature standard deviation. Table 2 shows the mean and standard deviation values used for feature scaling.

TABLE 2
Input features used for predicting nocturnal hypoglycemia. Nomenclature used
in this table is as follows: GROC, glucose rate of change; CV, coefficient
of variation of CGM; MIN, minimum CGM; MAX, maximum CGM; LBGI, low blood
glucose index; HBGI, high blood glucose index; CONGA, continuous overlapping
net glycemic action; STDGROC, standard deviation of the glucose rate of
change; TIR, percentage time in range 70-180 mg/dL; TBR[54|70], percentage
time below range <54 mg/dL | <70 mg/dL; TAR[180|250],
percentage time above range >180 mg/dL | >250 mg/dL.

| | IMPUTATION | | STANDARD |
|---|---|---|---|
| FEATURES | VALUE | MEAN | DEVIATION |
| GLUCOSE FEATURES | | | |
| GROC-LONGEST-GLUCOSE- | 1.12 | 1.20 | 0.59 |
| RISE@DAYTIME_PRIOR | | | |
| GROC-LONGEST-GLUCOSE- | −0.92 | −1.02 | 0.55 |
| DROP@DAYTIME_PRIOR | | | |
| CURRENT-GLUCOSE@AT_BEDTIME | 159.50 | 173.18 | 69.68 |
| GROC-LONGEST-GLUCOSE- | 1.12 | 1.21 | 0.61 |
| RISE@9 H_PRIOR | | | |
| LBGI@24 H_PRIOR | 0.23 | 0.55 | 0.92 |
| GROC-LONGEST-GLUCOSE- | −0.95 | −1.08 | 0.62 |
| DROP@9 H_PRIOR | | | |
| MAX@DAYTIME_PRIOR | 280.00 | 281.13 | 71.44 |
| MAX@9 H_PRIOR | 260.00 | 265.03 | 71.76 |
| MAX@4 H_PRIOR | 229.00 | 236.56 | 73.41 |
| TAR180@24 H_PRIOR | 0.39 | 0.40 | 0.28 |
| MIN@30 MIN_PRIOR | 150.00 | 163.62 | 68.17 |
| TAR250@24 H_PRIOR | 0.08 | 0.16 | 0.2 |
| LBGI@9 H_PRIOR | 0.12 | 0.5 | 0.98 |
| HBGI@24 H_PRIOR | 8.04 | 9.88 | 8.2 |
| TAR180@15 H_PRIO | 0.4 | 0.41 | 0.29 |
| MIN@DAYTIME_PRIOR | 79 | 86.36 | 32.36 |
| HBGI@15 H_PRIOR | 8.23 | 10.21 | 8.59 |
| TAR180@12 H_PRIOR | 0.41 | 0.42 | 0.29 |
| MIN@9 H_PRIOR | 88 | 98.1 | 40.12 |
| MIN@3 H_PRIOR | 118 | 130.85 | 56.91 |
| HBGI@9 H_PRIOR | 7.96 | 10.38 | 9.24 |
| TAR180@9 H_PRIOR | 0.4 | 0.42 | 0.31 |
| GROC-LONGEST-GLUCOSE- | 0.98 | 1.09 | 0.64 |
| RISE@4 H_PRIOR | | | |
| HBGI@3 H_PRIOR | 6.49 | 10.37 | 11.22 |
| TAR250@9 H_PRIOR | 0.05 | 0.16 | 0.23 |
| HBGI@6 H_PRIOR | 7.59 | 10.45 | 9.93 |
| TAR180@30 MIN_PRIOR | 0 | 0.4 | 0.47 |
| MIN-GLUCOSE-LATTER- | 119.5 | 128.81 | 46.46 |
| HALF@NIGHTTIME_WEEK_PRIOR | | | |
| TAR180@6 H_PRIOR | 0.4 | 0.42 | 0.33 |
| TAR180@4 H_PRIOR | 0.38 | 0.42 | 0.37 |
| GROC-LONGEST-GLUCOSE- | −0.89 | −1.04 | 0.67 |
| DROP@4 H_PRIOR | | | |
| LBGI@4 H_PRIOR | 0 | 0.49 | 1.29 |
| TBR70@24 H_PRIOR | 0 | 0.02 | 0.04 |
| TAR250@30 MIN_PRIOR | 0 | 0.15 | 0.34 |
| LBGI@DAYTIME_PRIOR | 0.2 | 0.5 | 0.86 |
| CONGA2 H@24 H_PRIOR | 56.86 | 58.9 | 22.5 |
| TAR250@4 H_PRIOR | 0 | 0.16 | 0.28 |
| STDROGC@24 H_PRIOR | 1.05 | 1.08 | 0.35 |
| CONGA2 H@15 H_PRIOR | 61.96 | 64.69 | 26.37 |
| CONGA4 H@DAYTIME_PRIOR | 69.75 | 74.1 | 33.52 |
| CONGA1 H@15 H_PRIOR | 44.36 | 46.02 | 17.14 |
| CONGA3 H@DAYTIME_PRIOR | 68.74 | 72.28 | 31.03 |
| GROC-LONGEST-GLUCOSEDROP_{—} | 120.00 | 126.17 | 46.14 |
| DURATION_MINUTES@24 H_PRIOR | | | |
| CV@24 H_PRIOR | 0.29 | 0.29 | 0.09 |
| GROC-LONGEST-GLUCOSERISE_{—} | 95 | 99.4 | 34.29 |
| DURATION_MINUTES@24 H_PRIOR | | | |
| CONGA1 H@9 H_PRIOR | 43.14 | 45.43 | 19.04 |
| CONGA2 H@9 H_PRIOR | 57.56 | 61.92 | 29.45 |
| STDROGC@9 H_PRIOR | 1.15 | 1.2 | 0.44 |
| GROC-LONGEST-GLUCOSERISE_{—} | 75 | 78.63 | 30.89 |
| DURATION_MINUTES@9 H_PRIOR | | | |
| TREND@1 H_PRIOR | −0.09 | −0.09 | 0.78 |
| TBR70@9 H_PRIOR | 0 | 0.02 | 0.04 |
| TBR54@24 H_PRIOR | 0 | 0 | 0.02 |
| CONGA4 H@9 H_PRIOR | 55.1 | 61.52 | 33.48 |
| GROC-LONGEST-GLUCOSEDROP_{—} | 85 | 91.28 | 37 |
| DURATION_MINUTES@9 H_PRIOR | | | |
| CV@9 H_PRIOR | 0.25 | 0.26 | 0.1 |
| STDROGC@3 H_PRIOR | 0.96 | 1.04 | 0.53 |
| GROC-LONGEST-GLUCOSERISE_{—} | 50 | 55.47 | 27.6 |
| DURATION_MINUTES@4 H_PRIOR | | | |
| CV@3 H_PRIOR | 0.15 | 0.17 | 0.1 |
| GROC-LONGEST-GLUCOSEDROP_{—} | 55 | 63.49 | 32.04 |
| DURATION_MINUTES@4 H_PRIOR | | | |
| TBR70@30 MIN_PRIOR | 0 | 0.02 | 0.11 |
| TBR70@3 H_PRIOR | 0 | 0.02 | 0.06 |
| GROC-LONGEST-GLUCOSERISE_{—} | 680 | 705.1 | 371.36 |
| TIMESINCE_MINUTES@24 H_PRIOR | | | |
| GROC-LONGEST-GLUCOSEDROP_{—} | 560 | 549.3 | 250.07 |
| TIMESINCE_MINUTES@DAYTIME_PRIOR | | | |
| GROC-LONGEST-GLUCOSEDROP_{—} | 225 | 222.34 | 95.02 |
| TIMESINCE_MINUTES@6 H_PRIOR | | | |
| GROC-LONGEST-GLUCOSERISE_{—} | 325 | 321.55 | 136.6 |
| TIMESINCE_MINUTES@9 H_PRIOR | | | |
| TBR54@1 H_PRIOR | 0 | 0 | 0.04 |
| TBR54@4 H_PRIOR | 0 | 0 | 0.02 |
| GROC-LONGEST-GLUCOSEDROP_{—} | 155 | 152.28 | 64.04 |
| TIMESINCE_MINUTES@4 H_PRIOR | | | |
| GROC-LONGEST-GLUCOSERISE_{—} | 125 | 119.77 | 47.79 |
| TIMESINCE_MINUTES@3 H_PRIOR | | | |
| PHYSICAL ACTIVITY FEATURES | | | |
| REPORTED | 0 | | |
| DURATION | 0.00 | 1.84 | 12.02 |
| TYPE_CARDIO | 0 | | |
| TYPE_STRENGTH | 0 | | |
| INTENSITY_LOW | 0 | | |
| INTENSITY_MEDIUM | 0 | | |
| TIMING_SIN | 0 | | |
| TIMING_COS | 0 | | |
| DEMOGRAPHICS | | | |
| AGE | 23.00 | 28.13 | 19.07 |
| SEX_F | 0 | | |
| SEX_M | 0 | | |

No sampling or data augmentation methods were applied to address dataset imbalance due to the relatively moderate nature of this imbalance (i.e., ˜100:10 in the training dataset).³² Furthermore, the use of imbalance correction techniques may not consistently lead to substantial enhancements in accuracy and might yield poorly calibrated models that overestimate the probability of infrequent events³³ such as nocturnal hypoglycemia in T1D.

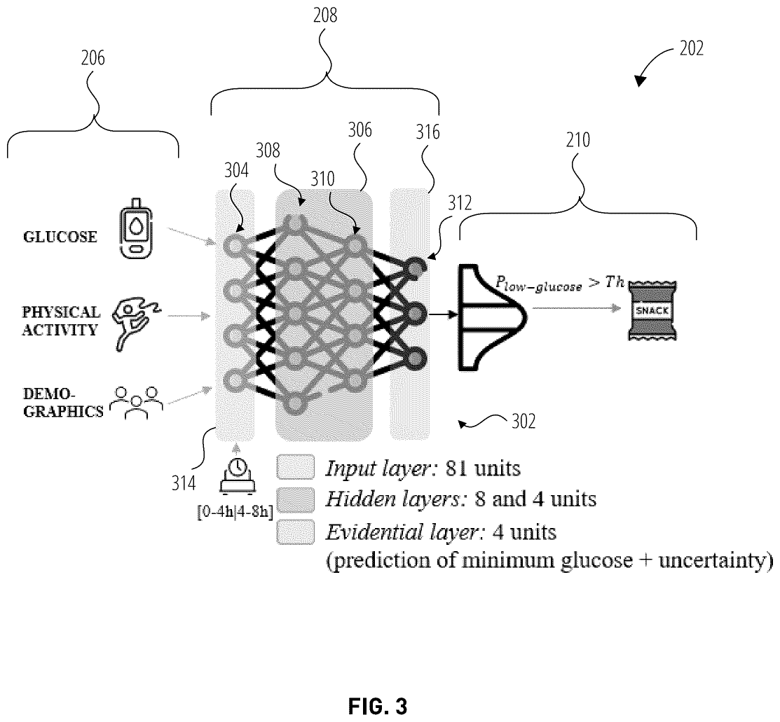

Set of features 220 are then passed to trained ENN model 208, an example of which is described in more detail with reference to FIG. 3. Trained ENN model 208 uses this set of features 220 to calculate or predict a minimum nocturnal glucose level and an associated predicted probability 222 (uncertainty) of a nocturnal hypoglycemia event. In the example shown here, trained ENN model 208 is optimized to predict the minimum nocturnal glucose and its associated uncertainty (probability) 222 at a first and second time interval 224 (0-4 hours and 4-8 hours in this example).

In Smart Snack intervention recommendation algorithm 210, predicted probability 222 of a nocturnal hypoglycemia event is compared to a threshold probability value 226 to determine whether alert 204 should be presented to the user (e.g., via display screen 114, FIG. 1) prior to bedtime, and if so, also presents recommended Smart Snack 228 to mitigate the risk of a dangerous hypoglycemic excursion.

Smart Snack intervention: Consumption of a bedtime snack was recommended if there was a predicted high likelihood of nocturnal hypoglycemia. The snack content was designed in collaboration with clinicians to address both the severity and timing of hypoglycemia. The carbohydrate content of the snack varied from 15 to 30 g based on the predicted minimum nocturnal glucose. Rapid-acting carbohydrates were recommended when the likelihood of a hypoglycemia event exceeded a target threshold in the first four hours of sleep. A mixed macronutrient snack with protein, fat, and fiber was recommended to delay carbohydrate absorption and address hypoglycemia if trained ENN model 208 predicted a high likelihood of hypoglycemia that again exceeded a threshold in the latter half of the night. The thresholds for likelihood of hypoglycemia were determined through hyperparameter tuning during training.

In Silico evaluation of hypoglycemia prediction and Smart Snack intervention: The effect of the Smart Snack intervention was evaluated in a virtual population of 20 adults with T1D. We matched 20 participants from a 4-arm automated insulin delivery study that was published previously³⁷ to the most similar virtual adults in the Oregon Health & Science University virtual population using total daily insulin requirements and body weight.³⁸ Each virtual adult participated in an in silico 3-arm crossover study with 77 days per arm, including (1) standard basal and bolus regimens without Smart Snack intervention, (2) Smart Snack intervention based on ENN predictions, and (3) Smart Snack intervention using knowledge of actual overnight glucose values (the Oracle forecast method).¹⁷ Varying insulin dosing regimens were imposed on the participants to reflect the impact of the intervention in a more real-world situation. The final in silico dataset reflects 4620 days of virtual participant outcomes. Glycemic outcomes are based on virtual participant CGM data and reported for the overnight period defined as 11 pm-7 am, as well as for the 24 h following bedtime at 11 pm. Bedtime was fixed for the in silico trialise.

As used herein, the term “evidential neural network (ENN)” refers to a neural network configured to output parameters of a probability distribution that represent both a predicted value and a measure of associated predictive uncertainty. In some embodiments, the evidential neural network produces parameters (see, e.g., γ, υ, α, β in Equation 1, below) of a Normal Inverse-Gamma distribution, which together characterize a predicted mean, variance, and corresponding uncertainty (“evidence”) for a regression task. The ENN may be implemented using a fully connected feed-forward architecture, convolutional layers, or other deep-learning architectures, trained using a loss function incorporating both a data-fitting term and an evidential regularization term that penalizes overconfident erroneous predictions.

FIG. 3, for instance, shows prediction algorithm 202 with trained ENN model 208 shown in greater detail. In this example, trained ENN model 208 includes a fully connected ENN 30234 that predicts, at bedtime, the minimum nocturnal glucose for a given time frame along with associated predictive uncertainty. The model architecture of fully connected ENN 302 includes an input layer of 81 units 304 (for simplicity, only four are shown) that are also called nodes or neurons, a series of hidden layers 306 of eight units 308 (five shown) and four units 310, and an evidential output layer of four units 312 (three shown) described below (see, e.g., γ, υ, α, β in Equation 1, below).

Evidential Regression: in this example, fully connected ENN 302 was optimized to predict, at bedtime, the minimum nocturnal glucose along with associated predictive uncertainty. Although a universally accepted definition of nocturnal hypoglycemia has not been established, in this example we conservatively defined nocturnal hypoglycemia as occurring when minimum nocturnal glucose levels drop below 70 mg/dL during either the initial or later four-hour period following bedtime. Evidential regression has been used previously for personalized short-term glucose prediction.³⁵ The evidential regression framework presented by Amini et al.³⁴ was adopted in this work and is described in the paragraphs immediately below.

Given a dataset with input features dᵢ and targets y_{I}, the evidential regression framework assumes the targets are independent and identically distributed from yᵢ˜N(μ, σ²) with unknown mean and variance, and probabilistically estimates an evidential posterior distribution q(μ, σ²)=p(μ, σ²|yᵢ). This is done by placing priors over the likelihood parameters, such that ˜N(γ, σ²/υ) and σ²˜Γ⁻¹(α, β). Γ(⋅) is the gamma function, and γ∈R, υ>0, α>1, and β>0. The resulting evidential distribution q(μ, σ²) is assumed to be factorizable q(p, σ²)=q(μ)q(σ²) and therefore, it takes the form of the Gaussian conjugate prior following a Normal Inverse-Gamma distribution p(μ, σ²|γ, υ, α, β). For continuous targets such as the minimum nocturnal glucose, evidential models directly learn the parameters γ, υ, α, β using maximum likelihood estimation for minimizing a two-component loss function: (1) negative log-likelihood plus (2) evidential regularization that penalizes prediction errors (Equation 1). The parameters γ, υ, α, β define full distributions of likelihood parameters and capture the uncertainty in the model's prediction.

$$ℒ = - log ( {y}_{i} \vert γ , υ , α , β ) + λ \vert {y}_{i} - γ \vert ( 2 υ + α ) , \qquad Equation 1$$

where λ is a hyperparameter to control evidential regularization

The evidential regression model outputs four values corresponding to the estimated parameters γ, υ, α, B. The predicted target ŷ and total uncertainty u are given in Equations 2 and 3, respectively. The probability of nocturnal hypoglycemia is calculated as (x<70 mg/dL), X˜N(γ, u²).

$$\hat{y} = γ \qquad Equation 2$$

$$u = \sqrt{\frac{β}{α - 1} ( 1 + \frac{1}{υ} )} \qquad Equation 3$$

The architecture of fully connected ENN 302 (input layer: 81 units, hidden layers: 8 and 4 units, evidential output layer: 4 units) and training hyperparameters (starting learning rate: 2.4×10⁻³, batch size: 128, and evidential penalty λ: 2.6×10⁻³) were found via Bayesian optimization using 5-fold participant-level cross-validation on the training dataset. One output of the ENN (γ) is an estimation of the minimum glucose overnight. The three other outputs of the ENN (υ, α, β) were used to estimate the uncertainty of the minimum glucose estimate. An indicator variable was used to specify whether the minimum overnight glucose is in hours 0-4 or 4-8 relative to bedtime. Table 3 summarizes these model architecture layer parameters and training parameters, along with a summary of input features, input variables, and model outputs.

TABLE 3
High-level description of the disclosed prediction-based decision
support tool for prevention of nocturnal hypoglycemia.

| | |
|---|---|
| FULLY CONNECTED MODEL | |
| ARCHITECTURE | |
| INPUT LAYER | [INPUT]: 81 INPUTS |
| HIDDEN DENSE LAYERS | [L1]: 8 neurons |
| | [L2]: 4 NEURONS |
| EVIDENTIAL LAYER | [OUTPUT]: γ, υ, α, β |
| MODEL TRAINING PARAMETERS | |
| STARTING LEARNING RATE | [LR]: 2.4 × 10⁻³ |
| EVIDENTIAL PENALTY | [Λ]: 2.4 × 10⁻³ |
| BATCH SIZE | [BATCH]: 128 SAMPLES |
| | PER BATCH |
| INPUT FEATURES (SEE TABLE 2 | |
| FOR DETAILED LIST OF | |
| INPUT FEATURES) | |
| GLUCOSE | GLUCOSE CONTROL AND |
| PHYSICAL ACTIVITY | STATISTICS TYPE, |
| | DURATION, INTENSITY, |
| | AND TIMING |
| DEMOGRAPHICS | AGE AND BIOLOGICAL SEX |
| INPUT VARIABLE | |
| Prediction time frame: Binary variable | [FIRST_HALF]: 1 |
| to indicate whether the algorithm | [SECOND_HALF]: 0 |
| return predictions of nocturnal | |
| hypoglycemia occurring in the first | |
| half or second half of the night. | |
| MODEL OUTPUTS | |
| PREDICTED TARGET | [γ]: Predicted minimum |
| | overnight glucose for a given |
| | prediction frame (first half or |
| | second half of the night) |
| | [u]: Total prediction uncertainty |
| | calculated as: |
| PREDICTED UNCERTAINTY | $u = \sqrt{\frac{β}{α - 1} ( 1 + \frac{1}{υ} )}$ |

Model parameters were optimized using the Adam optimizer.³⁶ Early stopping was used to prevent overfitting in the cross-validation runs. This means that training was halted before completing a usually very large set number of training epochs if the performance of the model as measured by the area under the receiver operating characteristic curve (AUROC) on the validation subset started to degrade.

Nocturnal hypoglycemia prediction evaluation metrics: AUROC, sensitivity, and specificity as well as the area under the precision-recall curve (AUPRC) were used to assess prediction accuracy. Risk thresholds for predicting nocturnal hypoglycemia were selected by maximizing the harmonic mean of the specificity and sensitivity on the training dataset to balance type I (false positives) and type II (false negatives) errors. In this application, both type I and type II errors are undesirable. Type I errors will result in high glucose levels and possibly weight gain if unnecessary treatments are indicated based on false positive predictions while type II errors will result in possibly preventable nocturnal hypoglycemia events.

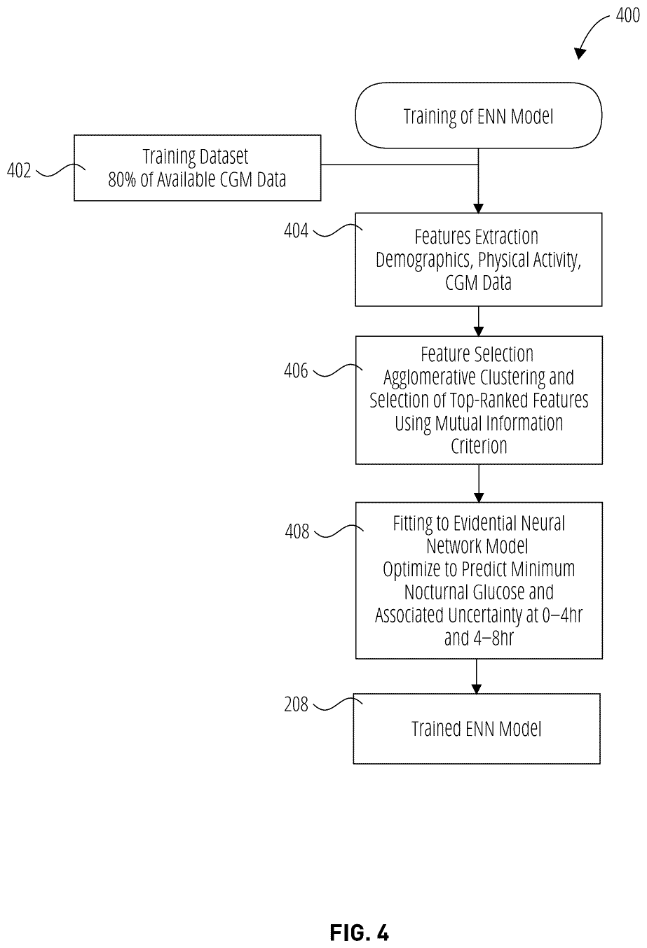

FIG. 4 shows an example training procedure 400 for training trained ENN model 208 (FIG. 2). As shown, training procedure 400 entails providing a training dataset 402 to a feature extraction module 404, which is configured to extract a large set of candidate features from the CGM data as well as a smaller set of features from the physical activity and demographic data. Table 2 provides examples of extracted candidate features that may be used in the procedure for training a trained ENN model 208.

These candidate features are passed to feature selection module 406 to generate a subset of top-ranked features to be used for training and, eventually, prediction. In the example shown here, feature selection module 406 employs an agglomerative clustering approach with a mutual information criterion to rank-order the extracted candidate features received from feature extraction module 404, but skilled persons will appreciate that other approaches for feature selection may be used. The subset of top-ranked features are then passed to an evidential regression module 408 to train trained ENN model 208. As noted, trained ENN model 208 is optimized to predict the minimum nocturnal glucose and its associated uncertainty (probability) at a first and second time interval using the subset of top-ranked features drawn from training dataset 402. The final output of training procedure 400 is a trained ENN model 208.

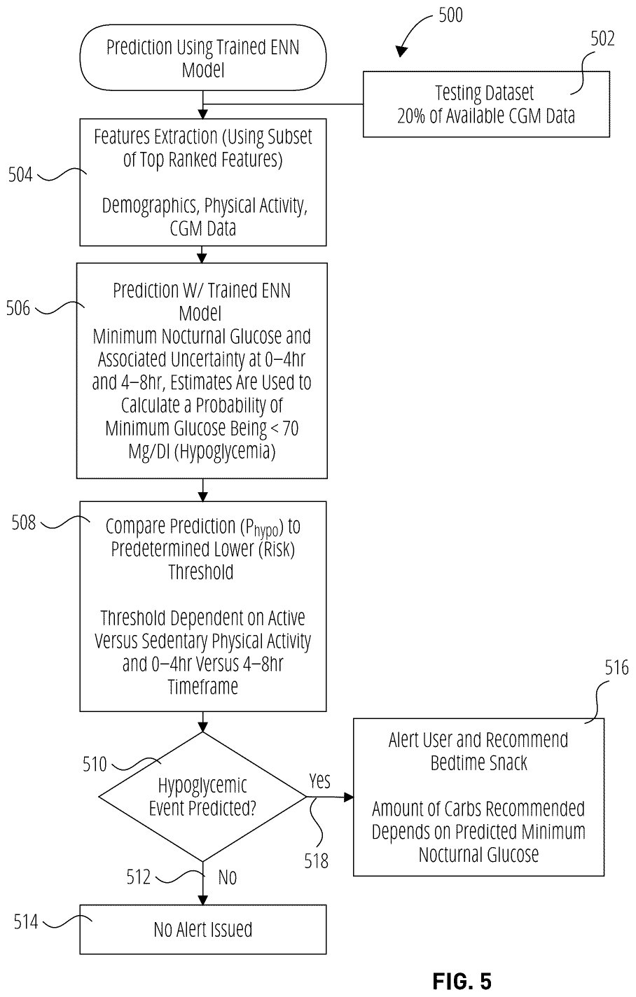

FIG. 5 shows an example procedure 500 for testing and making predictions using the trained ENN model 208 (FIG. 2-FIG. 4). In the example shown here, a testing dataset 502 is provided as input to a feature extraction module 504, which uses a subset of the top-ranked features identified in feature selection module 406 of training procedure 400. The extracted subset of top-ranked features are then passed to a prediction module 506, which employs trained ENN model 208 to predict a minimum nocturnal glucose value and associated uncertainty (probability) at a first and/or second time interval (0-4 hours and 4-8 hours in this example).

The predicted minimum nocturnal glucose value and associated uncertainty estimates are used to calculate a probability of minimum glucose being <70 mg/dL (hypoglycemia). That information is passed to a threshold comparison module 508, which employs a physical activity- and timeframe-dependent probability threshold level to determine whether a hypoglycemic event prediction is valid at decision point 510. If the hypoglycemic event prediction is deemed invalid 512, procedure 500 proceeds to not issue an alert 514. However, if the hypoglycemic event prediction satisfies the specified threshold and is deemed valid, procedure 500 proceeds to nocturnal hypoglycemia alert module 516 and alerts the user that an adverse hypoglycemic event is likely occur during the upcoming sleep session.

In some embodiments, nocturnal hypoglycemia alert module 516 also prescribes an appropriate bedtime snack for the user to consume before going to sleep to avoid or dampen the magnitude and duration of an adverse hypoglycemic event during the night. For instance, the amount of carbs recommended depends on the predicted minimum nocturnal glucose value.

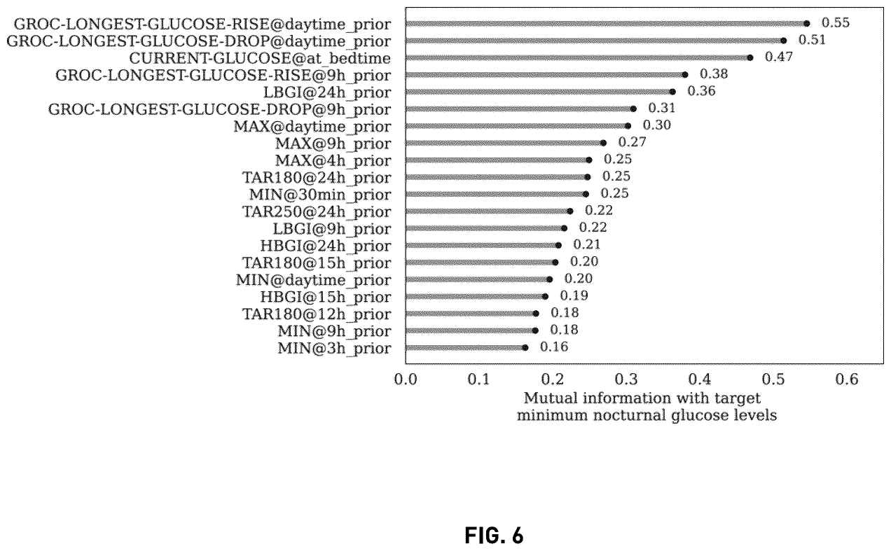

FIG. 6 shows the top 20 glucose features used to train the prediction algorithm ranked by their average mutual information (MI) with the target minimum nocturnal glucose during the first half and latter half of the night. Nomenclature for the features shown in FIG. 6 is as follows: GROC, glucose rate of change; MIN, minimum CGM glucose; MAX, maximum CGM glucose; LBGI, low blood glucose index; HBGI, high blood glucose index; TAR [180|250], percentage time above range >180 mg/dL|>250 mg/dL. PA features included exercise type, duration, intensity, and timing. Demographic features were age and biological sex. Table 2 shows an example of input features considered, from which the top 20 features of FIG. 6 were identified.

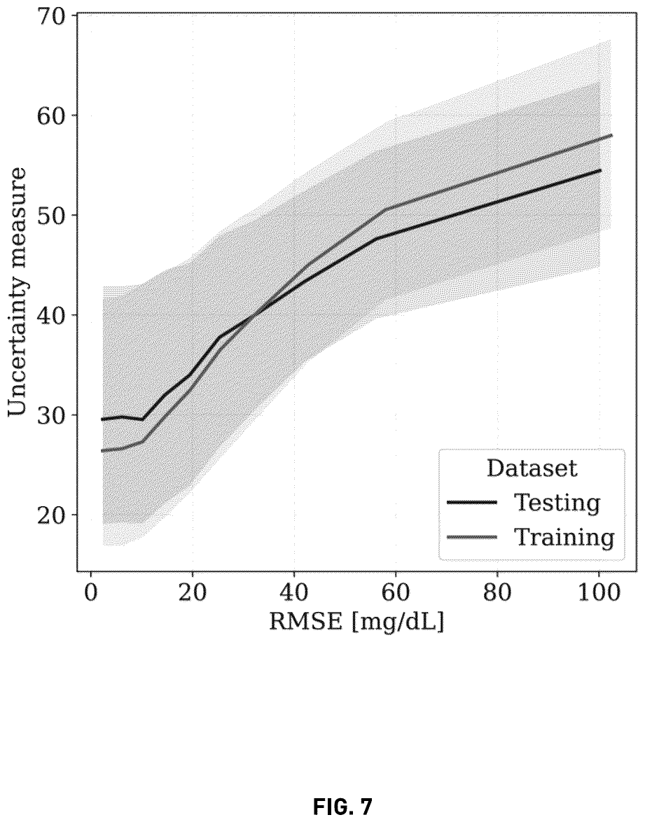

FIG. 7 shows the association between prediction error (RMSE, root mean square error) and evidential uncertainty for the training (blue) and the testing (black) datasets. Median (bold line) and interquartile range of uncertainty measure (shadow area) are also shown. These data demonstrate that uncertainty increased with increased error in the estimation of the minimum nocturnal glucose as expected.

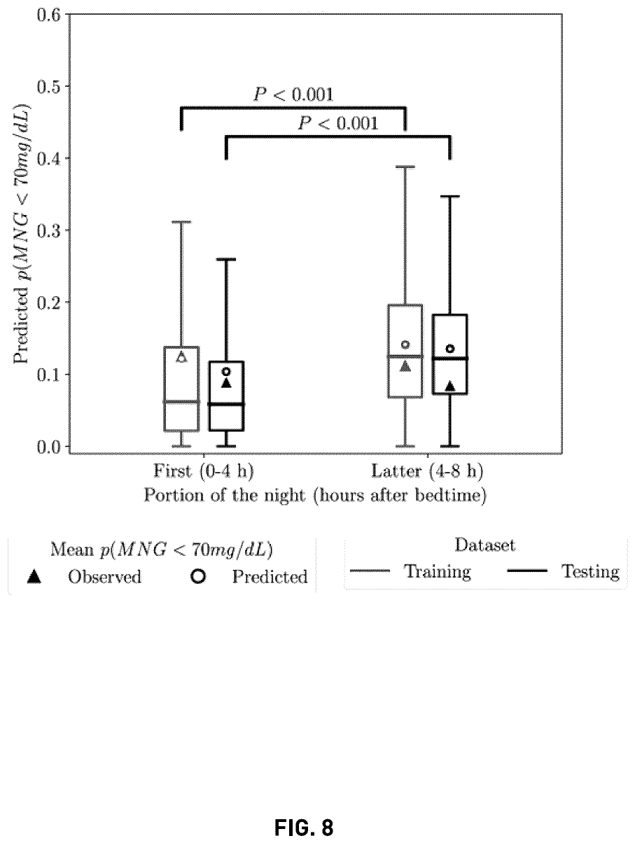

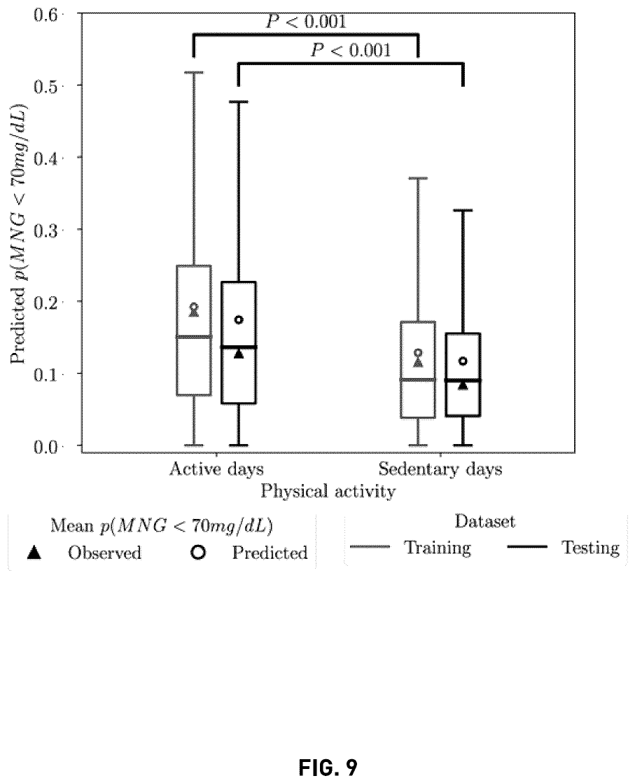

FIG. 8 is a box plot showing predicted probability of nocturnal hypoglycemia stratified by portion of the night, and FIG. 9 shows predicted probability of nocturnal hypoglycemia stratified by active versus sedentary days. Predicted probability of nocturnal hypoglycemia is higher during the latter half of the night compared with the first part of the night (training: 0.14 vs 0.12, testing: 0.14 vs 0.10, P<0.001). Predicted probability of nocturnal hypoglycemia is higher for nights following active days (training: 0.19 vs 0.13, testing: 0.17 vs 0.12, P<0.001). In the vertical axis of FIG. 8 and FIG. 9, MNG is an abbreviation for “minimum nocturnal glucose.”

The ENN predicts the probability of hypoglycemia during the 0-4 h and the 4-8 h after bedtime. FIG. 8 shows that the algorithm forecasted a higher likelihood of hypoglycemia during the latter half of the night compared with the first part of the night (P<0.001). Higher likelihood of hypoglycemia was also forecasted on physically active days (i.e., days with labeled exercise events) compared with sedentary days (P<0.001) as presented in FIG. 9.

There was 46.2% and 41.7% higher predicted probability of nocturnal hypoglycemia following physically active days relative to sedentary days in the training and testing data, respectively. Because of these differences, different thresholds were applied for predicting nocturnal hypoglycemia during the first half versus the latter half of the night, and for active versus sedentary days. The threshold

$${p}_{TH}^{( S , 1 )} = 0.112$$

for nocturnal hypoglycemia prediction during the first half of the night and

$${p}_{TH}^{( S , 2 )} = 0.167$$

for nocturnal hypoglycemia prediction during the latter half of the night on sedentary days. Similarly, for active days, the threshold

$${p}_{TH}^{( A , 1 )} = 0.159$$

for nocturnal hypoglycemia prediction during the first half of the night and

$${p}_{TH}^{( A , 2 )} = 0.211$$

for nocturnal hypoglycemia prediction during the latter half of the night. Customizing these thresholds through hyperparameter tuning resulted in a 25% improvement in median participant-level specificity (0.77 vs 0.52) at the expense of a 5% drop in participant-level sensitivity (0.81 vs 0.86) in predicting nocturnal hypoglycemia on active days.

To illustrate the operation of the algorithm, let us consider that a person is going to sleep at 11:00 μm and runs the algorithm to determine whether they should take a snack before bedtime to avoid hypoglycemia. At 11:00 pm, a feature vector is constructed including features derived from CGM, PA data, and demographics. Additionally, an input variable indicating that the model is tasked to return predictions for the first half of the night is used. The resulting feature vector and the indicator input variable are used to predict the minimum nocturnal glucose and the associated predicted uncertainty. Following this prediction, the algorithm generates a probability value p for the likelihood of the minimum nocturnal glucose falling below 70 mg/dL. If PA has been recorded during the day, an alert indicating a high likelihood of nocturnal hypoglycemia during the first half of the night will be triggered if

$$p > {p}_{T H}^{( A , 1 )}$$

and a bedtime snack recommendation will be provided. If the alert is triggered for the first half of the night, no further predictions are performed. However, if

$$p \leq {p}_{T H}^{( A , 1 )}$$

a new prediction will be initiated using the previously calculated feature vector, but with the indicator input variable set such that the model returns predictions for the second half of the night. If the probability of nocturnal hypoglycemia

$$p > {p}_{T H}^{( A , 2 )} ,$$

an alert for high likelihood of nocturnal hypoglycemia during the second half of the night is triggered and a bedtime snack recommendation will be provided. But if

$$p \leq {p}_{T H}^{( A , 2 )} ,$$

the risk of nocturnal hypoglycemia is considered to be low enough to not trigger an alert. This same process is applied for sedentary days using corresponding probability thresholds

$${p}_{TH}^{( S , 1 )} and {p}_{TH}^{( S , 2 )} .$$

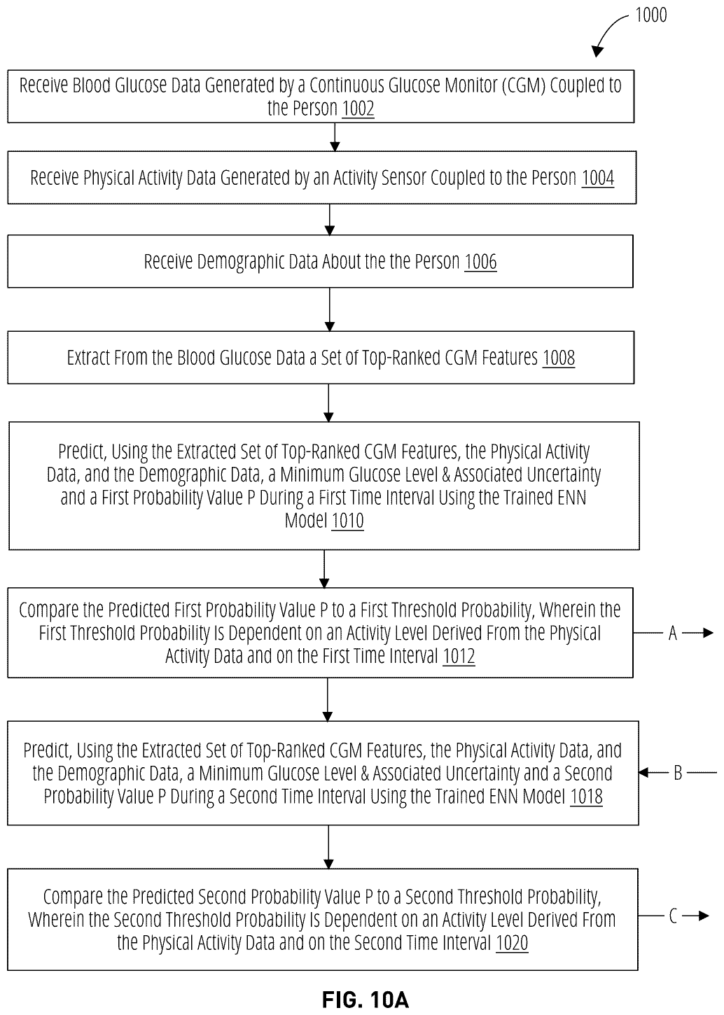

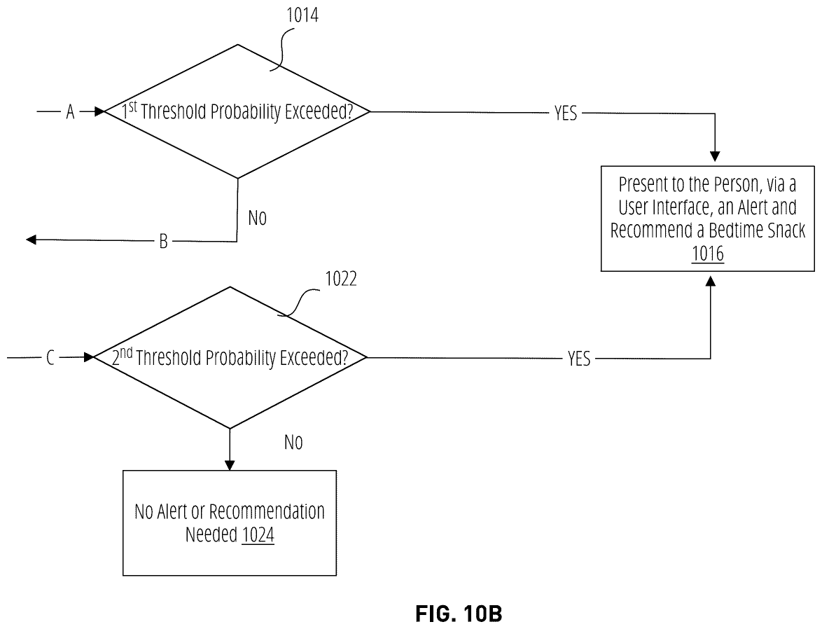

FIG. 10A shows a process 1000 of predicting, for a person using NHAIS 100 (FIG. 1), the likelihood of experiencing a nocturnal hypoglycemic event and whether an alert and snack recommendation should be presented. In this example, process 1000 and any presentation of alerts and snack recommendations is performed by computing device 102 (FIG. 1) configured with app 104. Process 1000 in this example mirrors the algorithm operation described in the paragraphs above.

In block 1002, process 1000 receives glucose data generated by a CGM coupled to the person. At block 1004, process 1000 receives physical activity data from physical activity sensor 110, and at block 1006, process 1000 receives the person's demographic information. At block 1008, process 1000 extracts from blood glucose data a set of top-ranked CGM data features (see, for example, FIG. 6), and forms a feature vector from the set of top-ranked CGM data features, the physical activity data, and demographic information. At block 1010 of process 1000, the feature vector and an input variable indicating that the model is tasked to return predictions for a first time interval (the first half of the night) is used to predict a minimum glucose level and associated predicted uncertainty using the trained EEN model. At block 1012 the minimum glucose level and associated predicted uncertainty are used to generate a first probability value p1 for the likelihood of the minimum nocturnal glucose falling below a specified hypoglycemic level (e.g., 70 mg/dL).

With reference to FIG. 10B, this probability value p1 is compared to a first threshold probability corresponding to the first time interval (first half of the night) and activity level. At block 1014, if probability value p1 exceeds the first threshold probability, the person is presented with an alert and bedtime snack recommendation at block 1016. If an alert is triggered in this first time interval, no further predictions are calculated.

However, if the first threshold probability is not exceeded at block 1014 a new prediction is initiated at block 1018 (FIG. 10A). Using the previously calculated feature vector but with the indicator input variable set such that the model returns predictions for the second half of the night, the trained EEN model is used to predict a new minimum glucose level and associated predicted uncertainty. At block 1020 the new minimum glucose level and associated predicted uncertainty are used to generate a second probability value p2 for the likelihood of the minimum nocturnal glucose falling below a specified hypoglycemic level (e.g., 70 mg/dL).

Referring to FIG. 10B, this probability value p2 is compared to a second threshold probability corresponding to the second time interval (second half of the night) and activity level. At block 1022, if probability value p2 exceeds the second threshold probability, an alert is triggered at block 1016 and bedtime snack recommendation is presented. But if probability value p2 does not exceed the second threshold probability at block 1022, the risk of nocturnal hypoglycemia is considered to be low enough to not trigger an alert and snack recommendation so process 1000 proceeds to block 1024 and terminates.

In other embodiments, computing device 102 receives data and transmits it to a remote server for further processing. In that embodiment, the remote server performs the operations in process 1000 and returns any triggered alerts and snack recommendations to computing device 102. In still other embodiments, different processing steps are performed by each of computing device 102 and remote server, depending on the particular configuration.

Table 4 shows the higher performance of the proposed ENN algorithm when compared with Elastic Net,³⁸ Support Vector Regression,³⁹ and XGBOOST,⁴⁰ based on predictions made on the testing dataset which included the actual nocturnal glucose measurements that were used as ground truth. Comparator models were chosen such that various machine learning algorithms of different levels of complexity were evaluated, and the SVR model was included as the family of Support Vector Machine methods has been used in the past for predicting nocturnal hypoglycemia with promising results.^{16,17,19,22,23} For a more objective comparative assessment of the accuracy of the prediction methods, the ENN and all comparator models were trained and tested on the same datasets described herein in the Methods/Datasets section.

TABLE 4
Results of nocturnal hypoglycemia prediction
methods on the hold-out testing dataset.

| | | Models | | | |
|---|---|---|---|---|---|
| | | | Support | Extreme | Disclosed |
| Nocturnal | | | vector | gradient | evidential |
| hypoglycemia | Performance | Elastic | regression | boosting | neural |
| time frame | metric | Net | (SVR) | (XGBOOST) | network |
| First half of the | AUROC | 0.74 | 0.77 | 0.75 | 0.80 |
| night 0-4 hours | Sensitivity | 0.61 | 0.61 | 0.61 | 0.67 |
| after bedtime | Specificity | 0.75 | 0.79 | 0.77 | 0.77 |
| | AUPRC (Chance | 0.32 | 0.38 | 0.32 | 0.43 |
| | level = 0.09) | | | | |
| Latter half of the | AUROC | 0.64 | 0.67 | 0.64 | 0.71 |
| night 4-8 hours | Sensitivity | 0.51 | 0.52 | 0.51 | 0.58 |
| after bedtime | Specificity | 0.69 | 0.73 | 0.67 | 0.72 |
| | AUPRC (Chance | 0.17 | 0.20 | 0.18 | 0.22 |
| | level = 0.09) | | | | |
| Nocturnal | Sensitivity | 0.65 | 0.63 | 0.62 | 0.68 |
| Hypoglycemia | Specificity | 0.71 | 0.75 | 0.69 | 0.74 |
| regardless of | | | | | |
| event timing | | | | | |

The impact of the Smart Snack intervention in silico (mean±SD) is presented in Table 5. The statistical significance of the differences in glucose control outcomes was assessed using the Wilcoxon signed-rank test. ENN predictions in conjunction with the Smart Snack intervention resulted in 41% lower probability of nocturnal hypoglycemia relative to the no intervention baseline (14.0% vs 23.9%). Moreover, the ENN-based Smart Snack intervention significantly reduced % time-in-hypoglycemia as compared to no intervention, both in the overnight period (2.4% vs 7.4%) and during the 24 h following bedtime (1.8% vs 1.2%), with no change in % time-in-range. Using the Oracle forecasting method to recommend carbohydrates based on absolute knowledge of future nocturnal hypoglycemia resulted in comparable % time-in-hypoglycemia of 2.8%, and higher % time-in-range during the overnight period and the 24 h following bedtime given that the Oracle forecasting method does not produce Type I errors possibly leading to hyperglycemia.

TABLE 5
Evaluation of the Smart Snack intervention in silico.

| | | % Time-in- | | | | % Time-in- | |
|---|---|---|---|---|---|---|---|
| | | hypoglycemia | | % Time-in-range | | hyperglycemia | |
| | Probability | (<70 mg/dL) | | (70-180 mg/dL) | | (>180 mg/dL) | |
| | of nocturnal | Mean ± SD | | Mean ± SD | | Mean ± SD | |
| | hypoglycemia | | 24 | | 24 | | 24 |
| Study Arm | (<70 mg/dL) | Nighttime | hours | Nighttime | hours | Nighttime | hours |
| No intervention | 23.9 ± | 7.4 ± | 3.6 ± | 60.10 ± | 54.8 ± | 32.5 ± | 41.6 ± |
| | 14.1% | 7.0% | 2.3% | 9.7% | 10.0% | 11.8% | 10.3% |
| Evidential neural | 14.0 ± | 2.4 ± | 1.8 ± | 60.7 ± | 54.4 ± | 36.9 ± | 43.8 ± |
| network + Smart | 13.3%^{a} | 3.3%^{a} | 1.2%^{a} | 11.1%^{b} | 9.8%^{b} | 12.3%^{a,b} | 10.3%^{a,b} |
| Snack | | | | | | | |
| Oracle + Smart | 13.5 ± | 2.8 ± | 2.2 ± | 65.1 ± | 56.1 ± | 32.2 ± | 41.6 ± |
| Snack | 10.3% | 3.5% | 1.9% | 11.1% | 9.5% | 12.5% | 10.7% |

Statistical significance indicates p-value <0.05 as evaluated by Wilcoxon Signed-Rank test.
^{a}Statistically significant with respect to control arm.
^{b}Statistically significant with respect to oracle smart snack.

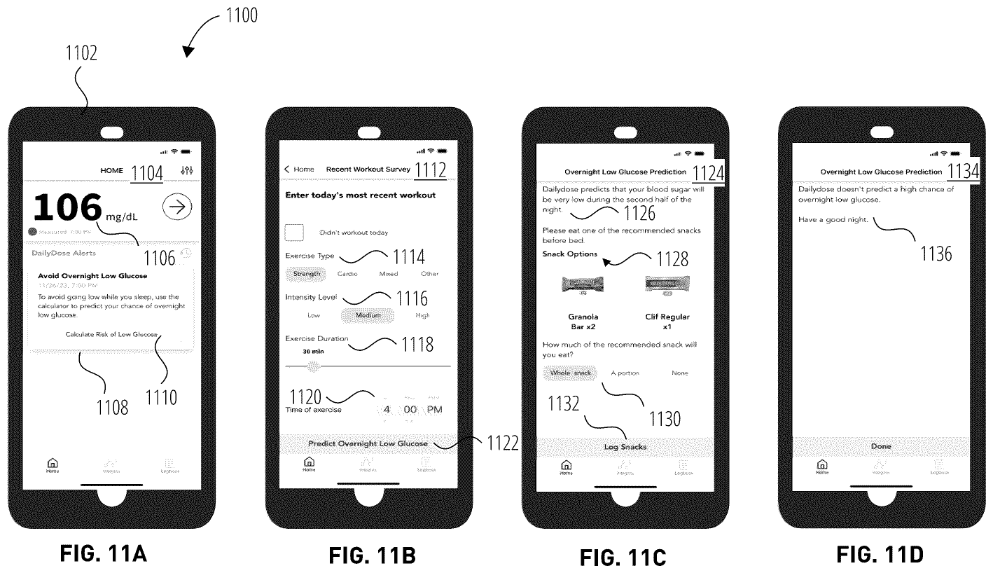

FIG. 11 illustrates example screenshots of a DailyDose Smart Snack decision-support application 1100 executed on a mobile device 1102. App 1100 is configured to integrate data from CGM devices, wearable activity sensors, and user-entered data to provide personalized nocturnal hypoglycemia prevention recommendations. In FIG. 11A, a home screen 1104 displays a current glucose value 1106 obtained from a CGM system and includes a DailyDose alert panel 1108 prompting the user to access a low-glucose-risk calculator 1110. Selection of the calculator launches a prediction workflow for assessing overnight hypoglycemia risk.

In FIG. 11B, a workout-survey screen 1112 allows the user to enter self-reported information describing their most recent physical activity, including exercise type 1114 (e.g., strength, cardio, mixed, or other), intensity level 1116, duration slider control 1118, and time-of-exercise input 1120. Once triggered 1122, these data are transmitted to a cloud-based prediction engine (not shown) that implements an uncertainty-aware ENN 208 (FIG. 2) to compute the probability and timing of a nocturnal low-glucose event. Skilled persons will appreciate that the workout/activity data may also be obtained automatically via fitness tracking devices (e.g., a smart watch).

In FIG. 11C, when the neural-network model predicts a high probability of overnight hypoglycemia, a snack-recommendation interface 1124 presents a notification 1126 and one or more personalized snack options 1128. Each option corresponds to a carbohydrate content selected to address both the predicted severity and the expected timing of nocturnal low glucose. For example, a small snack (≈15 g carbohydrate) or large snack (≈30 g carbohydrate) may be suggested based on the predicted minimum overnight glucose.

When hypoglycemia is predicted within the first four hours of sleep, the algorithm recommends a fast-absorbing carbohydrate snack, such as a 4-oz juice cup (not shown). When hypoglycemia is predicted to occur during the latter portion of the night (e.g., 4-8 hours after sleep onset), the algorithm recommends a slow-absorbing mixed-macronutrient snack, for example a granola or protein bar having an approximate 4:2:1 ratio of carbohydrates, protein, and fat, and containing about 1-2 g of dietary fiber. The user may select an input 1130 indicating whether they consumed a whole snack or portion, after which the “Log Snacks” button 1132 records the entry.

In FIG. 11D, a prediction-result screen 1134 presents a notification 1136 indicating a low probability of overnight hypoglycemia. When the predicted risk is low, no snack recommendation is displayed, and the user may simply dismiss the screen.

DailyDose Smart Snack decision-support application 1100 thus provides a clinical intervention by (i) acquiring CGM-derived features, physical-activity metrics, and user survey inputs; (ii) applying an evidential neural-network model to estimate the probability and temporal profile of nocturnal low glucose (<70 mg/dL); and (iii) generating tailored snack recommendations to mitigate predicted overnight hypoglycemia. The user interface informs the participant whether the predicted event is expected during the first or second half of the night and enables compliance logging for subsequent analysis.

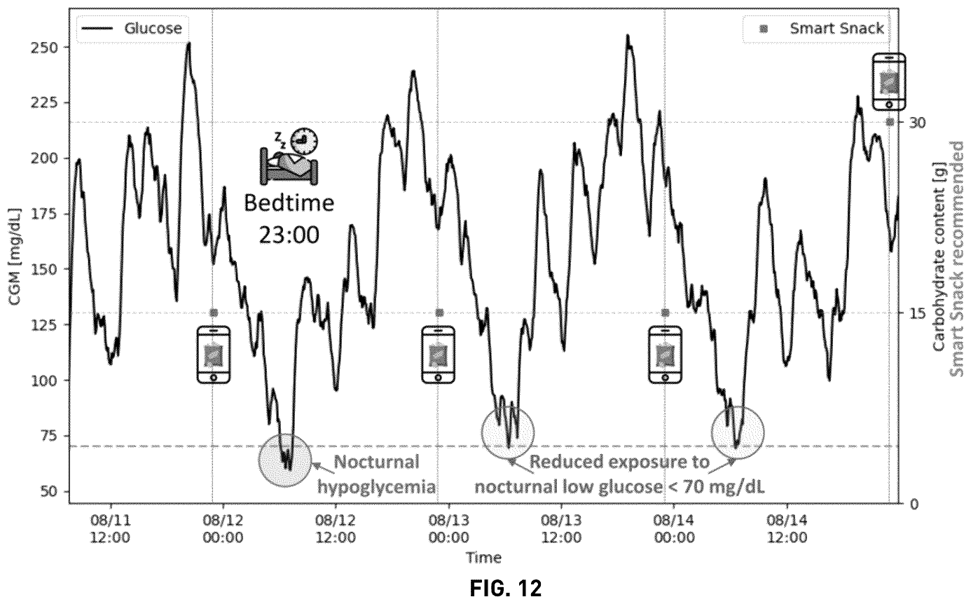

FIG. 12 shows, in the context of a real-world clinical application over four days, how the prediction-based recommendation system disclosed herein is integrated, in a particular embodiment, into a mobile health app for MDI users.⁴⁵ As discussed, this integration involves automated processing of an individual's glucose measurements from a CGM sensor and PA data from a smartwatch or fitness tracker paired with the smartphone running a decision support app. The core functionality of the module for prevention of overnight low glucose encompasses preemptive alerts and personalized bedtime Smart Snack suggestions. These recommendations are triggered when there is a heightened predicted probability of a nocturnal hypoglycemia event. For two days, FIG. 12 shows how such a clinical application is effective in reducing exposure to nocturnal low glucose by use of the Smart Snack intervention in reducing nocturnal hypoglycemia for individuals with T1D using MDI therapy.

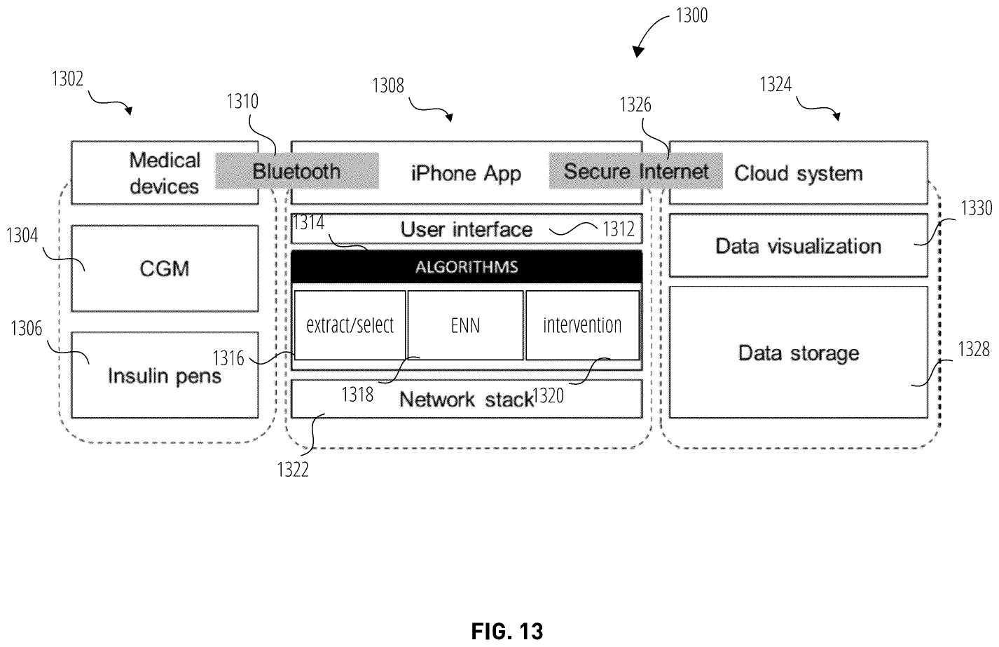

FIG. 13 shows a cloud-connected diabetes management system 1300, which supports insulin delivery optimization by processing CGM data, insulin, and exercise data to enhance real-time glucose control and treatment interventions. System 1300 integrates with both automated insulin delivery (AID) systems and connected insulin pens (both of which may be referred to generally as connected insulin delivery systems), adjusting insulin dosing dynamically to optimize glucose outcomes. By leveraging real-time physiological data and predictions, system 1300 improves glucoregulatory hormone dosing by enabling automated updates and optimization of the glucoregulatory hormone dosage parameters, making it a comprehensive, multi-functional diabetes management platform, making it a comprehensive, multi-functional diabetes management platform. More generally, connected insulin delivery systems are types of connected glucoregulatory hormone delivery systems for delivery of one or more of insulin, glucagon, and pramlintide.

In AID system embodiments, system 1300 automatically adjusts insulin dosing by transmitting optimized insulin delivery parameters to automated pumps, ensuring real-time closed-loop control. In connected insulin pen (or smart pen) embodiments, system 1300 provides insulin dosing adjustments based on predicted glucose trends, allowing users to administer the optimal insulin dose using the optimized settings of the insulin dosing system.

Specifically, system 1300 includes a medical device 1302—which may be a CGM 1304, a connected insulin pen 1306, or an automated insulin pump—a user's software application 1308 (e.g., a smartphone, smartwatch, or other smart device app), and a cloud-based software application 1308 running on associated computing devices. Medical device 1302 communicates real-time glucose and insulin delivery data via a personal area network (PAN) connection 1310 (e.g., Bluetooth) to software application 1308.

Software application 1308 generates a user interface 1312 that provides real-time feedback (e.g., recommended Smart Snack 228, FIG. 2) and insulin adjustment insights derived from system 1300. FIG. 13 depicts core algorithms 1314, including feature extraction/selection 1316, ENN 1318, and Smart Snack intervention 1320 that help avoid nocturnal hypoglycemia For completeness, software application 1308 also includes lower-layer OS components such as network stack 1322, which manage connectivity and data synchronization.

Software application 1324 receives data from application 1308 via a secure internet connection 1326, processes it using its own algorithms (such as cloud-hosted versions pf algorithms 1314), and stores it in data storage 1328 for advanced analytics. The processed data is then used to generate data visualizations 1330 on user interface 1312, allowing users and healthcare providers to track trends, optimize dosing strategies, and improve long-term glucose management.

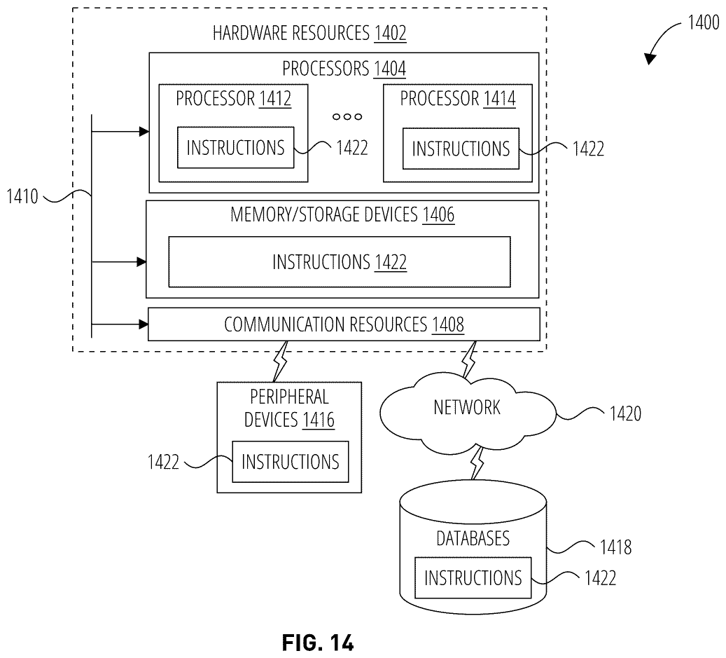

FIG. 14 is a block diagram illustrating components 1400, according to some example embodiments, able to read instructions from a machine-readable or computer-readable medium (e.g., a non-transitory machine-readable storage medium), and perform any one or more of the methods discussed herein (e.g., process 1000, FIG. 10A and FIG. 10B). For example, hardware resources 1402 may be embodied in a smartwatch, server, tablet computer, or patient-connected device that provides the ability to measure a physiological signal, provide some analysis of that signal, transmit information about that signal, and/or support a user interface to provide information about that signal. This includes an equivalent functional combination, for example a watch that can measure a physiological signal and transmit the data to a computer (including a smartphone), where the computer provides analysis and user interface functions.

Specifically, FIG. 14 shows a diagrammatic representation of hardware resources 1402 including one or more processors 1404 (or processor cores), one or more memory/storage devices 1406, and one or more communication resources 1408, each of which may be communicatively coupled via a bus 1410.

Processors 1404 (e.g., a central processing unit (CPU), a reduced instruction set computing (RISC) processor, a complex instruction set computing (CISC) processor, a graphics processing unit (GPU), a digital signal processor (DSP) such as a baseband processor, an application specific integrated circuit (ASIC), another processor, or any suitable combination thereof) may include, for example, a processor 1412 and a processor 1414.

Memory/storage devices 1406 may include main memory, disk storage, or any suitable combination thereof. Memory/storage devices 1406 may include, but are not limited to any type of volatile or non-volatile memory such as dynamic random access memory (DRAM), static random-access memory (SRAM), erasable programmable read-only memory (EPROM), electrically erasable programmable read-only memory (EEPROM), Flash memory, solid-state storage, etc.

Communication resources 1408 may include interconnection or network interface components or other suitable devices to communicate with one or more peripheral devices 1416 or one or more databases 1418 via a network 1420. For example, communication resources 1408 may include wired communication components (e.g., for coupling via a Universal Serial Bus (USB)), cellular communication components, NFC components, Bluetooth® components (e.g., Bluetooth® Low Energy), Wi-Fi® components, and other communication components.

Instructions 1422 may comprise software, a program, an application, an applet, an app, or other executable code for causing at least any of processors 1404 to perform any one or more of the methods discussed herein (e.g., process 1000, FIG. 10A and FIG. 10B). In some embodiments, instructions 1422 include aspects of software running on NHAIS 100, including an ENN-based hypoglycemia prediction model stored in memory/storage devices 1406.

Instructions 1422 may reside, completely or partially, within at least one of processors 1404 (e.g., within the processor's cache memory), memory/storage devices 1406, or any suitable combination thereof. Furthermore, any portion of instructions processors 1404 may be transferred to hardware resources 1402 from any combination of peripheral devices 1416 or databases 1418. Accordingly, memory of processors 1404, memory/storage devices 1406, peripheral devices 1416, and databases 1418 are examples of computer-readable and machine-readable media.

The disclosed embodiments demonstrate the performance of an ENN framework and explores the clinical impact of utilizing this algorithm with a Smart Snack approach to recommend carbohydrates before bed, after both sedentary days and physically active days, if indicated. The ENN algorithm performs better than baseline methods in terms of AUROC and sensitivity in predicting nocturnal hypoglycemia. The SVR model achieved the highest specificity (1%-2% higher than the specificity of the ENN). However, comparator models lack the capability to estimate predictive uncertainty. The proposed algorithm provides uncertainty assessments that are well correlated with the prediction error (FIG. 7), enabling the identification of scenarios where model predictions might be inaccurate. For instance, the uncertainty of the ENN predictions is optimized to be higher in scenarios when the model is applied to individuals with features (e.g., demographic or glucose dynamics features) that significantly differ from those individuals whose data were used for model development.

The existing literature describes alternative approaches for estimating prediction uncertainties in neural networks using Bayesian techniques, such as mean-variance estimation,⁴¹ Monte Carlo dropout,⁴² and ensemble methods.⁴³ These methods generate prediction distributions by introducing noise to the model inputs, randomly dropping out a proportion of the neurons in a neural network or building ensembles of models with diverse parameters. However, the framework of evidential learning adopted in this study is different from these Bayesian strategies. Instead of placing a prior distribution on model inputs or parameters, it directly places a prior distribution on the likelihood function of the target variable, allowing a more flexible representation of uncertainty, as the likelihood function can capture both epistemic and aleatoric uncertainty.³⁴

The results in this disclosure suggest that the ENN algorithm might help avoid 68% of nocturnal hypoglycemic events, with a specificity of 74% (i.e., 1.8 false positives per week). As expected, the accuracy of the algorithm is lower in predicting hypoglycemia that occurs in the latter half of the night (Table 4).

The prediction algorithm was equally effective in predicting nocturnal risk after both sedentary and physically active days. It is also worth noting that physically active days appeared to significantly increase the predicted risk for nocturnal hypoglycemia, which is consistent with other research in clinical data observations for youth living with T1D who did or did not perform afternoon exercise.⁴⁴

In previous work on nocturnal hypoglycemia prediction, the inventors have described an SVR algorithm that was trained and validated using data collected from people with T1D on sensor-augmented pump therapy (SVR_{PUMP}). That previous work tested the SVR_{PUMP} on the MDI testing dataset. SVR_{PUMP} achieved sensitivity and specificity of 0.65 and 0.64 in predicting nocturnal hypoglycemia, respectively. The optimal glucose features found on data from pump users are comparable to MDI users. However, the SVR model trained with MDI (SVR_{MDI}) data and additional PA features yielded an overall sensitivity of 0.63 and a specificity of 0.75, which was worse than the ENN (Table 4). Although there was a slight decrease in sensitivity for the SVR_{MDI}, the additional features and the training data resulted in an 11% improvement in specificity, compared with the SVR_{PUMP}. This demonstrates the importance of training the model on MDI data if the algorithm is to be used by people on MDI therapy.

The above embodiments further explored the impact of utilizing the ENN algorithm and Smart Snack in an in silico clinical trial. It was shown that consuming a Smart Snack in response to the ENN predictions reduced both nocturnal hypoglycemia and hypoglycemia the following day; and this reduction was comparable to that of the Oracle method, which had full knowledge of nocturnal hypoglycemia prior to bedtime. These findings lend support for use of the ENN as an effective decision support tool to help people living with T1D avoid overnight hypoglycemia.

As shown in the disclosed embodiments, advances in evidential machine learning were leveraged to develop a model capable of predicting the probability and timing of nocturnal hypoglycemia. Information on risk and timing are key for making informed decisions regarding proactive measures to avoid nocturnal hypoglycemia.

The evidential learning approach provides an estimation of predictive uncertainty, enabling identification of scenarios where the model is likely to fail; and therefore, improving the models' robustness and reliability. This system, in conjunction with the Smart Snack algorithm, significantly reduced hypoglycemia overnight and in the following day in an in silico trial.

Skilled persons will now appreciate that many changes may be made to the details of the above-described embodiments without departing from the underlying principles of the invention. The scope of the present invention should, therefore, be determined only by claimed features and equivalents.

### REFERENCES

1. Nathan D M; DCCT/EDIC Research Group. The diabetes control and complications trial/epidemiology of diabetes interventions and complications study at 30 years: overview. Diabetes Care 2014; 37(1): 9-16.
2. Nathan D M, Cleary P A, Backlund J-Y C, et al.; Diabetes Control and Complications Trial/Epidemiology of Diabetes Interventions and Complications (DCCT/EDIC) Study Research Group. Intensive diabetes treatment and cardiovascular disease in patients with type 1 diabetes. N Engl J Med. 2005; 353(25): 2643-2653.
3. MacDonald M J. Postexercise late-onset hypoglycemia in insulin dependent diabetic patients. Diabetes Care 1987; 10(5): 584-588.
4. McMahon S K, Ferreira L D, Ratnam N, et al. Glucose requirements to maintain euglycemia after moderate-intensity afternoon exercise in adolescents with type 1 diabetes are increased in a biphasic manner. J Clin Endocrinol Metab. 2007; 92(3): 963-968.
5. Sandoval D A, Guy D L A, Richardson M A, Ertl A C, Davis S N. Effects of low and moderate antecedent exercise on counterregulatory responses to subsequent hypoglycemia in type 1 diabetes. Diabetes 2004; 53(7):1798-1806.
6. Sandoval D A, Guy D L A, Richardson M A, Ertl A C, Davis S N. Acute, same-day effects of antecedent exercise on counterregulatory responses to subsequent hypoglycemia in type 1 diabetes mellitus. Am J Physiol Endocrinol Metab. 2006; 290(6): E1331-E1338.
7. Hedrington M S, Davis S N. Sexual dimorphism in glucose and lipid metabolism during fasting, hypoglycemia, and exercise. Front Endocrinol (Lausanne). 2015; 6(61).
8. Mosquera-Lopez C, Ramsey K L, Roquemen-Echeverri V, Jacobs P G. Modeling risk of hypoglycemia during and following physical activity in people with type 1 diabetes using explainable mixed-effects machine learning. Comput Biol Med. 2023; 155:106670.
9. Brunton S A. Nocturnal hypoglycemia: answering the challenge with long-acting insulin analogs. Medscape Gen Med. 2007; 9(2): 38.
10. Juvenile Diabetes Research Foundation Continuous Glucose Monitoring Study Group, et al. Continuous glucose monitoring and intensive treatment of type 1 diabetes. N Engl J Med. 2008; 359(14):1464-1476.
11. McGill J B, Ahmann A. Continuous glucose monitoring with multiple daily insulin treatment: outcome studies. Diabetes Technol Ther. 2017; 19 (S3):S3-S12.
12. McAuley S A, Vogrin S, Lee M H, et al.; Australian JDRF Closed-Loop Research Group. Less nocturnal hypoglycemia but equivalent time in range among adults with type 1 diabetes using insulin pumps versus multiple daily injections. Diabetes Technol Ther. 2021; 23(6): 460-466.
13. Kalergis M, Schiffrin A, Gougeon R, Jones P J H, Yale J-F. Impact of bedtime snack composition on prevention of nocturnal hypoglycemia in adults with type 1 diabetes undergoing intensive insulin management using lispro insulin before meals. Diabetes Care 2003; 26(1): 9-15.
14. Campbell M D, Walker M, Bracken R M, et al. Insulin therapy and dietary adjustments to normalize glycemia and prevent nocturnal hypoglycemia after evening exercise in type 1 diabetes: a randomized controlled trial. BMJ Open Diabetes Res Care. 2015; 3(1):e000085
15. Riddell M C, Gallen I W, Smart C E, et al. Exercise management in type 1 diabetes: a consensus statement. Lancet Diabetes Endocrinol. 2017; 5(5): 377-390.
16. Bertachi A, Vi˜nals C, Biagi L, et al. Prediction of nocturnal hypoglycemia in adults with type 1 diabetes under multiple daily injections using continuous glucose monitoring and physical activity monitor. Sensors 2020; 20(6):1705.
17. Mosquera-Lopez C, Dodier R, Tyler N S, et al. Predicting and preventing nocturnal hypoglycemia in type 1 diabetes using big data analytics and decision theoretic analysis. Diabetes Technol Ther. 2020; 22(11): 801-811.
18. Jensen M H, Dethlefsen C, Vestergaard P, Hejlesen O. Prediction of nocturnal hypoglycemia from continuous glucose monitoring data in people with type 1 diabetes: a proof-of-concept study. J Diabetes Sci Technol. 2020; 14(2): 250-256.
19. Guemes A, Cappon G, Hernandez B, et al. Predicting quality of overnight glycaemic control in type 1 diabetes using binary classifiers. IEEE J Biomed Health Inform. 2020; 24(5):1439-1446.
20. Calhoun P, Levine R A, Fan J. Repeated measures random forests (RMRF): identifying factors associated with nocturnal hypoglycemia. Biometrics 2021; 77(1): 343-351.
21. Berikov V B, Kutnenko O A, Semenova J F, Klimontov V V. Machine learning models for nocturnal hypoglycemia prediction in hospitalized patients with type 1 diabetes. J Pers Med. 2022; 12(8):1262.
22. Parcerisas A, Contreras I, Delecourt A, et al. A machine learning approach to minimize nocturnal hypoglycemic events in type 1 diabetic patients under multiple doses of insulin. Sensors 2022; 22(4):1665.
23. Afentakis I, Unsworth R, Herrero P, Oliver N, Reddy M, Georgiou P. Development and validation of binary classifiers to predict nocturnal hypoglycemia in adults with type 1 diabetes. J Diabetes Sci Technol. 2023:19322968231185796.
24. Gillingham M B, Li Z, Beck R W, et al. Assessing mealtime macronutrient content: patient perceptions versus expert analyses via a novel phone app. Diabetes Technol Ther. 2021; 23(2): 85-94.
25. Riddell M C, Li Z, Gal R L, et al.; T1DEXI Study Group. Examining the acute glycemic effects of different types of structured exercise sessions in type 1 diabetes in a real-world setting: the Type 1 Diabetes and Exercise Initiative (T1DEXI). Diabetes Care 2023; 46(4): 704-713.
26. Kovatchev B P. Metrics for glycaemic control—from HbA1c to continuous glucose monitoring. Nat Rev Endocrinol. 2017; 13(7): 425-436.
27. Battelino T, Alexander C M, Amiel S A, et al. Continuous glucose monitoring and metrics for clinical trials: an international consensus statement. Lancet Diabetes Endocrinol. 2023; 11(1): 42-57.
28. Willett P, El-Hamdouchi A. Comparison of hierarchic agglomerative clustering methods for document retrieval. Comput J. 1989; 32(3): 220-227.
29. Rousseeuw P J. Silhouettes: a graphical aid to the interpretation and validation of cluster analysis. J Comput Appl Math. 1987; 20:53-65.
30. Tibshirani R. Regression shrinkage and selection via the Lasso. J R Stat Soc Ser B Methodol. 1996; 58(1): 267-288.
31. Zou H, Hastie T. Regularization and variable selection via the elastic net. J R Stat Soc Ser B Stat Methodol. 2005; 67(2): 301-320.
32. Hasanin T, Khoshgoftaar T M, Leevy J L, Bauder R A. Severely imbalanced Big Data challenges: investigating data sampling approaches. J Big Data.2019; 6(1):107.
33. Van Den Goorbergh R, Van Smeden M, Timmerman D, Van Calster B. The harm of class imbalance corrections for risk prediction models: illustration and simulation using logistic regression. J Am Med Inform Assoc. 2022; 29(9):1525-1534.
34. Amini A, Schwarting W, Soleimany A, Rus D. Deep evidential regression. In: Proceedings of the 34th Conference on Advances in Neural Information Processing Systems (NeurIPS 2020). MIT Press; 2020. Accessed Jan. 27, 2023.
35. Zhu T, Li K, Herrero P, Georgiou P. Personalized blood glucose prediction for type 1 diabetes using evidential deep learning and meta-learning. IEEE Trans Biomed Eng. 2023; 70(1):193-204.
36. Kingma D P, Ba J. Adam: a method for stochastic optimization. arXiv. https://doi.org/10.48550/arXiv.1412.6980 Jan. 29, 2017, preprint: not peer reviewed.
37. Castle J R, El Youssef J, Wilson L M, et al. Randomized outpatient trial of single- and dual-hormone closed-loop systems that adapt to exercise using wearable sensors. Diabetes Care 2018; 41(7):1471-1477.
38. Resalat N, Youssef J E, Tyler N, Castle J, Jacobs P G. A statistical virtual patient population for the glucoregulatory system in type 1 diabetes with integrated exercise model. PLOS One 2019; 14(7): e0217301.
39. Drucker H, Burges C J C, Kaufman L, Smola A, Vapnik V. Support vector regression machines. In: Proceedings of the 9th Conference on Advances in Neural Information Processing Systems (NIPS 1996). MIT Press; 1996. Accessed Feb. 9, 2023.
40. Chen T, Guestrin C. XGBoost: a scalable tree boosting system. Proceedings of the 22nd ACM SIGKDD International Conference on Knowledge Discovery and Data Mining, in KDD '16. Association for Computing Machinery; 2016:785-794.
41. Nix D A, Weigend A S. Estimating the mean and variance of the target probability distribution. Proceedings of 1994 IEEE International Conference on Neural Networks (ICNN'94). IEEE; 1994:55-60. vol. 1.
42. Gal Y, Ghahramani Z. Dropout as a Bayesian approximation: representing model uncertainty in deep learning. Proceedings of The 33rd International Conference on Machine Learning. PMLR; 2016:1050-1059. Accessed Aug. 8, 2023.
43. Hansen L K, Salamon P. Neural network ensembles. IEEE Trans Pattern Anal Machine Intell. 1990; 12(10): 993-1001.
44. Tsalikian E, Mauras N, Beck R W, et al. Impact of exercise on overnight glycemic control in children with type 1 diabetes mellitus. J Pediatr. 2005; 147(4): 528-534.
45. Castle J R, Wilson L M, Tyler N S, et al. Assessment of a decision support system for adults with type 1 diabetes on multiple daily insulin injections. Diabetes Technol Ther. 2022; 24(12): 892-897.

What is claimed is:

1. A computer-implemented method for reducing likelihood of a person experiencing nocturnal hypoglycemia, the method comprising:
extracting a plurality of features including glucose-derived features from continuous glucose monitor (CGM) data, activity-derived features from physical-activity data, and demographic-derived features associated with the person;
inputting the plurality of features into a neural network configured to output parameters of a probability distribution representing both a predicted minimum overnight glucose value and an associated predictive uncertainty;
determining, based on output of the neural network, whether a probability that the person's glucose level will fall below a specified hypoglycemic level within a specified time period during an upcoming sleep session meets or exceeds a predetermined risk threshold; and
in response to determining that the probability meets or exceeds the predetermined risk threshold indicative of a predicted hypoglycemia event, causing a display, on a user interface of a mobile computing device, of a personalized bedtime snack recommendation configured to allow the person to avoid the predicted hypoglycemia event.

2. The method of claim 1, further comprising receiving, via the user interface, an input indicating whether the person consumed a recommended snack, and logging the input in a user profile stored in memory of the mobile computing device or of a remote server.

3. The method of claim 1, in which the neural network includes an input layer configured to receive the plurality of features, one or more hidden layers, and an output layer configured to generate parameters of a normal inverse-gamma distribution.

4. The method of claim 3, in which the neural network is trained to output parameters (γ, ν, α, β) of the normal inverse-gamma distribution for characterizing both the predicted minimum overnight glucose value and the associated predictive uncertainty.

5. The method of claim 1, in which the plurality of features includes at least one of:
a glucose-trend feature calculated over the hour preceding bedtime;
a rate-of-change feature; or
an activity-intensity feature derived from wearable device data or input into the user interface by the person.

6. The method of claim 1, wherein the specified time period comprises a temporal window corresponding to an early-night window of approximately 0 to 4 hours after bedtime or a late-night window of approximately 4 to 8 hours after bedtime.

7. The method of claim 6, in which the personalized bedtime snack recommendation is for a fast-absorbing snack, comprising simple carbohydrates, selected for the early-night window.

8. The method of claim 6, in which the personalized bedtime snack recommendation is for a slow-absorbing snack comprising complex carbohydrates, protein, and fat selected for the late-night window.

9. The method of claim 8, in which the slow-absorbing snack has an approximate 4:2:1 ratio of complex carbohydrates, protein, and fat, with 1-2 grams of dietary fiber.

10. The method of claim 1, further comprising identifying the personalized bedtime snack recommendation having a carbohydrate content between approximately 15 grams and 30 grams based on the predicted minimum overnight glucose value.

11. The method of claim 1, in which the personalized bedtime snack recommendation further specifies a portion size selected based on the predicted minimum glucose value.

12. The method of claim 1, further comprising receiving, by the mobile computing device, the CGM data acquired from a glucose sensor worn by the person.

13. The method of claim 1, further comprising receiving, by the mobile computing device, self-reported physical-activity data entered by the person through the user interface of a smartphone application.

14. The method of claim 1, further comprising receiving, by the mobile computing device, monitored physical-activity data acquired automatically by a wearable fitness tracker worn by the person and communicatively coupled with the mobile computing device.

15. The method of claim 1, further comprising computing a early-night probability that the person's glucose level will fall below the specified hypoglycemic level during an early-night window corresponding to approximately 0 to 4 hours after bedtime and a late-night probability that the person's glucose level will fall below the specified hypoglycemic level during a late-night window corresponding to approximately 4 to 8 hours after bedtime.

16. The method of claim 15, further comprising comparing each of the early-night probability and the late-night probability to corresponding predetermined risk thresholds, and determining that a predicted hypoglycemia event will occur when either of the probabilities meets or exceeds its corresponding predetermined risk threshold.

17. The method of claim 16, further comprising identifying a corresponding bedtime snack recommendation based on which of the probabilities meets or exceeds its corresponding predetermined risk threshold.

18. The method of claim 17, in which a fast-absorbing snack comprising simple carbohydrates is selected when the early-night probability meets or exceeds its threshold, and a slow-absorbing snack comprising complex carbohydrates, protein, and fat is selected when the late-night probability meets or exceeds its threshold.

19. The method of claim 16, in which the predetermined risk threshold for the early-night window is greater than the predetermined risk threshold for the late-night window to account for a higher physiological sensitivity to early-night glucose decline.

20. The method of claim 16, in which the predetermined risk thresholds for the early-night and late-night windows are independently defined based on physical-activity level and timeframe.

21. The method of claim 15, in which the neural-network output includes different probability distribution parameter values for each window, and the early-night and late-night probabilities are computed from corresponding parameter values.
